(19) United States
(12) Patent Application Publication
(10) Pub. No.: US 2013/0266625 A1
(43) Pub. Date: Oct. 10, 2013

(54) NANOPARTICLES BASED ON POLY (LACTIC GLYCOLIC) ACID FOR COSMETIC APPLICATIONS

(71) Applicants: Simon Benita, Tel Aviv (IL); Taher Nasser, Tur'an Village (IL); Nour Karra, Tel Aviv (IL); Amit Badihi, Jerusalem (IL)

(72) Inventors: Simon Benita, Tel Aviv (IL); Taher Nasser, Tur'an Village (IL); Nour Karra, Tel Aviv (IL); Amit Badihi, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 13/995,259

(22) PCT Filed: Jan. 24, 2012
(86) PCT No.: PCT/IL2012/050019

§ 371 Date: Jun. 18, 2013

Related U.S. Application Data
(60) Provisional application No. 61/435,640, filed on Jan. 24, 2011.
(60) Provisional application No. 61/435,674, filed on Jan. 24, 2011.

(51) Int. Cl. A61K 8/85; A61Q 19/00

(52) U.S. Cl. CPC A61K 8/85; A61Q 19/00
USPC 424/401; 514/178; 514/54

(57) ABSTRACT

On one hand, the present invention relates to cosmetic compositions comprising poly(lactic glycolic)acid (PLGA) nanoparticles for applications to the skin. On the other hand, it also concerns polymeric nanoparticles having on its surface a plurality of cosmetically active agents, each of said agents being associated to said nanoparticle via oleylcysteineamide, delivery systems for topical application based on said particles and cosmetic formulations comprising said particles.

### FIELD OF THE INVENTION

The present invention relates, in most general terms, to polymer based nanoparticles for the dermal delivery of cosmetic agents.

### BACKGROUND OF THE INVENTION

Dermal therapy is still a challenge due to the inability to bypass the skin and deliver sufficient amounts of agents, either hydrophilic or lipophilic, to the deep skin layers. The penetration and permeation of poorly absorbed active ingredients can be improved by the addition of specific enhancers to the formulation, by the use of colloidal delivery systems, especially nanoparticles. The benefits of nanoparticles in such applications have been shown recently in several scientific fields, but little is known about the potential penetration of nanoparticles through the different skin layers. Nanoparticles may exert biological effects, simply by virtue of their dimension (100 nm or less).

Encapsulation using nanoparticulate systems is an increasingly implemented strategy in drug targeting and delivery. Such systems have been proposed for topical administration to enhance percutaneous transport into and across the skin barrier. However, the mechanism by which such particulate formulations facilitate skin transport remains ambiguous. These nanometric systems present a large surface area, a property that renders them a very promising delivery system for dermal and transdermal delivery. Their small particle size ensures close contact with the stratum corneum and the ability to control the particle diameter may modulate the skin site deep layer localization [1].

In a recent study, confocal laser scanning microscopy (CLSM) was used to visualize the distribution of non-biodegradable, fluorescent, polystyrene nanoparticles (diameters 20 and 200 nm) across porcine skin. The surface images revealed that (i) polystyrene nanoparticles accumulated preferentially in the follicular openings, (ii) this distribution increased in a time-dependant manner, and (iii) the follicular localization was favored by the smaller particle size. Apart from follicular uptake, localization of nanoparticles in skin “furrows” was apparent from the surface images. However, cross-sectional images revealed that these non-follicular structures did not offer an alternative penetration pathway for the polymer vectors, which transport was clearly impeded by the stratum corneum [2].

Recently, lipid nanoparticles have shown a great potential as vehicles for topical administration of active substances, principally owing to the possible targeting effect and controlled release in different skin strata. Ketoprofen and naproxen loaded lipid nanoparticles were prepared, using hot high pressure homogenization and ultra sonication techniques, and characterized by means of photocorrelation spectroscopy and differential scanning calorimetry. Nanoparticle behavior on human skin was assessed, in vitro, to determine drug percutaneous absorption (Franz cell method) and in vivo to establish the active localization (tape-stripping technique) and the controlled release abilities (UVB-induced erythema model). Results demonstrated that the particles were able to reduce drug penetration, increasing, simultaneously, the permeation and the accumulation in the horny layer. A prolonged anti-inflammatory effect was observed in the case of drug loaded nanoparticles with respect to the drug solution. Direct as well as indirect evidences corroborate the early reports on the usefulness of lipid nanoparticles as carriers for topical administration, stimulating new and deeper investigations in the field [3].

Polymeric nanocapsules have also been proposed as carriers for active agents for topical application. Among the many advantages of such delivery systems is the ability of the polymeric shell to achieve sustained release of the active ingredient and increase the sensitive compounds, thus resulting in an improved therapeutic effect of dermatological formulations. Currently, several commercially available cosmetic products have incorporated nanoparticles for the encapsulation of vitamin A, rose extract and wheat germ oil [4].

Another very recent paper published by Wu et al. [5] shows that polystyrene and poly(methyl methacrylate) nanoparticles were not able to pass beyond the most superficial layers of the skin, i.e., Stratum Corneum, following a 6 hours topical application; even polystyrene nanoparticles as small as 30 nm were not able to penetrate beyond the Stratum Corneum. On the other hand, the hydrophobic compound encapsulated inside the nanoparticles was released and was able to diffuse across the deeper layers of the skin.

The fact that nanoparticles are retarded at the skin surface may be an advantage, since the active ingredient can be slowly released over a prolonged period and diffuse across the skin barrier, while the nanoparticles themselves will not be systemically translocated. Thus, the authors [5] suggest that the penetration of nanoparticles across intact skin seems unlikely to induce a systemic effect.

Nevertheless, health authorities are very attentive to the potential negative effects that may be induced by non biodegradable nanoparticles within and across the skin following topical application. In fact, starting November 2009, member states of the EU have adopted a single regulation to cosmetic products: this was in fact the first national legislation to incorporate rules relating to the use of nanomaterials in any cosmetic products [6]. According to this regulation, anyone who wishes to distribute a new nanomaterials containing product will be required to hand out to the European Commission safety information prior entry to the market. It should be stressed that these concerns are related to the use of non biodegradable nanoparticles, whereas, the use of nanoparticles that will be degraded in the skin over a reasonable period of time is not expected to elicit any adverse effect especially if the degradation products are safe.

In the 1970s, biodegradable polymers were suggested as appropriate drug delivery materials circumventing the requirement of polymer removal [7]. Aliphatic polyesters such as poly(ε-caprolactone) (PCL), poly(3-hydroxybutyrate) (PHB), poly(glycolic acid) (PGA), poly(lactic acid) (PLA) and its copolymers with glycolic acid i.e., poly(D,L-lactide-coglycolide) (PLGA) [8-11] have been widely used to formulate the controlled release devices. The reason why PLA and PLGA are polymers that are widely used in the preparation of micro and nanoparticles, lies in the fact that they are non-toxic, well tolerated by the human body, biodegradable and biocompatible [12-13]. PLA and PLGA are FDA approved polymers for subcutaneous and intramuscular injections.

The degradation process of PLGA, also known as bulk erosion, occurs by autocatalytic cleavage of the ester bonds through spontaneous hydrolysis into oligomers and D,L-lactic and glycolic acid monomers [14]. Lactic acid enters the tricarboxylic acid cycle and is metabolized and eliminated as CO₂ and water. Glycolic acid is either excreted unchanged in the urine or enters the Krebs cycle and is also eliminated as CO₂ and water.

Recently the suitability of biodegradable poly-lactic acid (PLA, MW 30 000) nanoparticles loaded with fluorescent dyes as carriers for transepidermal drug delivery was investigated in human skin explants using fluorescence microscopy, confocal laser scanning microscopy and flow cytometry [15]. The results showed that PLA particles penetrated into 50% of the vellus hair follicles, reaching a maximal depth corresponding to the entry of the sebaceous gland in 12-15% of all observed follicles. The accumulation of particles in the follicular ducts was accompanied by the release of dye to the viable epidermis and its retention in the sebaceous glands for up to 24 h. Kinetic studies in vitro as well as in skin explants revealed destabilization of the particles and significant release of incorporated dye occurred upon contact with organic solvents and the skin surface. According to the authors these results suggest that particles based on PLA polymers may be ideal carriers for hair follicle and sebaceous gland targeting.

### REFERENCES

[1] Alves M P, Scarrone A L, Santos M, Pohlmann A R and Guterres S S (2007) Human skin penetration and distribution of nimesulide from hydrophilic gels containing nanocarriers. International journal of pharmaceutics 341(1-2):215-220.
[2] Alvarez-Roman R, Naik A, Kalia Y N, Guy R H and Fessi H (2004) Skin penetration and distribution of polymeric nanoparticles. J Control Release 99(1):53-62.
[3] Puglia C, Blasi P, Rizza L, Schoubben A, Bonina F, Rossi C and Ricci M (2008) Lipid nanoparticles for prolonged topical delivery: an in vitro and in vivo investigation. International journal of pharmaceutics 357(1-2):295-304.
[4] Wu X, Price G J and Guy R H (2009b) Disposition of nanoparticles and an associated lipophilic permeant following topical application to the skin. Molecular pharmaceutics 6(5): 1441-1448.
[5] Wu X, Griffin P, Price G J and Guy R H (2009a) Preparation and in vitro evaluation of topical formulations based on polystyrene-poly-2-hydroxyl methacrylate nanoparticles. Molecular pharmaceutics 6(5):1449-1456.
[6] Bowman D M, van Calster G and Friedrichs S (2010) Nanomaterials and regulation of cosmetics. Nature nanotechnology 5(2):92.
[7] Jalil R and Nixon J R (1990) Biodegradable poly(lactic acid) and poly(lactide-co-glycolide) microcapsules: problems associated with preparative techniques and release properties. Journal of microencapsulation 7(3):297-325.
[8] Vert M, Schwach G, Engel R and Coudane J (1998) Something new in the field of PLA/GA bioresorbable polymers? J Control Release 53(1-3):85-92.
[9] Jain R A (2000) The manufacturing techniques of various drug loaded biodegradable poly(lactide-co-glycolide) (PLGA) devices. Biomaterials 21(23):2475-2490.
[10] Uhrich K E, Cannizzaro S M, Langer R S and Shakesheff K M (1999) Polymeric Systems for Controlled Drug Release. Chemical Reviews 99(11):3181-3198.
[11] Park T G (1995) Degradation of poly(lactic-co-glycolic acid) microspheres: effect of copolymer composition. Biomaterials 16(15):1123-1130.
[12] Pistner H, Bendix D R, Muhling J and Reuther J F (1993) Poly(L-lactide): a long-term degradation study in vivo. Part III. Analytical characterization. Biomaterials 14(4):291-298.
[13] Mordenti J, Thomsen K, Licko V, Berleau L, Kahn J W, Cuthbertson R A, Duenas E T, Ryan A M, Schofield C, Berger T W, Meng Y G and Cleland J (1999) Intraocular pharmacokinetics and safety of a humanized monoclonal antibody in rabbits after intravitreal administration of a solution or a PLGA microsphere formulation. Toxicol Sci 52(1):101-106.
[14] Li S (1999) Hydrolytic degradation characteristics of aliphatic polyesters derived from lactic and glycolic acids. Journal of biomedical materials research 48(3):342-353.
[15] Rancan F, Papakostas D, Hadam S, Hackbarth S, Delair T, Primard C, Verrier B, Sterry W, Blume-Peytavi U, Vogt A (2009) Investigation of polylactic acid (PLA) nanoparticles as drug delivery systems for local dermatotherapy. Pharm Res 26(8):2027-36.
[16] Chang L C, Caira M R and Guillory J K (1995) Solid state characterization of dehydroepiandrosterone. Journal of pharmaceutical sciences 84(10):1169-1179.
[17] Mitragotri S (2004) Breaking the skin barrier. Advanced drug delivery reviews 56(5):555-556.
[18] Tan G, Xu P, Lawson L B, He J, Freytag L C, Clements J D and John V T (2010) Hydration effects on skin microstructure as probed by high-resolution cryo-scanning electron microscopy and mechanistic implications to enhanced transcutaneous delivery of biomacromolecules. Journal of pharmaceutical sciences 99(2):730-740.
[19] Fessi H, Puisieux F, Devissaguet J P, Ammoury N and Benita S (1989) Nanocapsule formation by interfacial polymer deposition following solvent displacement. International Journal of Pharmaceutics 55 R1-R4.
[20] Methods On Glycoconjugates, Informa Healthcare publications, 1995, Andre Verbert, Chapter 4: Colorimetric Micro Methods, pp 29-30.
[21] Japanese patent application no. JP2010150151
[22] PCT publication no. WO 2010/091187

### SUMMARY OF THE INVENTION

The present invention is based on a novel approach for the construction of vehicles for delivery of cosmetic agents, which by themselves or in combination with various other active agents have the ability to penetrate the skin and induce a cosmetic/dermatological, non-systemic effect. Where the vehicles are associated with cosmetically active agents, they are capable of delivering sufficient amounts of the agents, either hydrophilic or lipophilic, to the deep skin layers to induce a topical non-systemic cosmetic effect. The vehicles of the invention are able to cross biological membranes, provide the ability to simultaneously deliver more than one cosmetically active agent to a desired site, in particular both hydrophobic and hydrophilic agents, and most importantly to deliver macromolecules which administration otherwise is impeded. As may be appreciated, known nanoparticulate delivery systems such as liposomes and nanoemulsions are limited in their ability, mainly because such systems cannot incorporate significant concentrations of hydrophilic macromolecules and/or enhance their penetration and prolonged residence time in the upper layers of the skin.

The nanoparticle vehicles of the invention possess long physicochemical shelf-life over long storage periods, as freeze-dried powders, which can maintain their initial properties upon reconstitution with the addition of purified or sterile water prior to use.

The invention disclosed herein is based on a nanoparticle which may be used per se, or may be modified to carry one or more cosmetically active agents. The nanoparticle employed in accordance with the invention is able, naked or comprising additional agents, to penetrate into a tissue barrier, e.g., skin to at least the 10 superficial epidermis layers, to a depth of at least 4-20 μm (micrometers). The nanoparticles biodegrade in the skin layer into which they penetrate and can thus, in addition to the effect that may be exerted by the associated agent, provide, e.g., hydration of the penetrable tissue by lactic acid and/or glycolic acid for a period of at least 24 hours, 72 hours, and even for a period of weeks.

Within the scope of the invention disclosed herein, the term “skin” refers to any region of a mammalian skin (human skin), including skin of the scalp, hair and nails. The skin region to which the composition of the invention may be applied, depends inter alia on parameters discussed herein.

It is thus the purpose of a first aspect of the invention to provide a cosmetic composition comprising poly(lactic glycolic) acid (PLGA) nanoparticles and a cosmetically acceptable carrier, the nanoparticle having an averaged diameter of at most 500 nm, the PLGA having an averaged molecular weight of between 2,000 and 20,000 Da.

In some embodiments, the nanoparticles polymer consists essentially of PLGA, namely, the nanoparticle backbone polymer is only PLGA and the active or non-active agents which it may be associated with, as further disclosed hereinbelow, may vary in accordance with embodiments of the invention.

In some embodiments, the PLGA has an averaged molecular weight of between 2,000 and 10,000 Da. In other embodiments, the PLGA has an averaged molecular weight of between 2,000 and 7,000 Da. In other embodiments, the PLGA has an averaged molecular weight of between 2,000 and 5,000 Da. In still further embodiments, the PLGA has an averaged molecular weight of between 4,000 and 20,000 Da, or between 4,000 and 10,000 Da, or between 4,000 and 5,000 Da. In still other embodiments, the PLGA has an averaged molecular weight of about 2,000, about 4,500, about 5,000, about 7,000, or about 10,000 Da.

As used herein, the “nanoparticle” employed in the cosmetic compositions of the invention is a particulate carrier, nanocapsule or a nanosphere, which is biocompatible and sufficiently resistant to chemical and/or physical destruction, such that a sufficient amount (concentration or population) of the nanoparticles remains substantially intact after administration into the human or animal body and for a sufficient time period to be able to reach the desired target tissue (organ). Generally, the nanoparticles are spherical in shape, having an averaged diameter of up to 500 nm. Where the shape of the particle is not spherical, the diameter refers to the longest dimension of the particle.

In some embodiments, the averaged diameter is between about 10 and 50 nm. In further embodiments, the averaged diameter is at least about 50 nm.

In some embodiments, the averaged diameter is between about 100 and 200 nm. In other embodiments, the averaged diameter is between about 200 and 300 nm. In further embodiments, the averaged diameter is between about 300 and 400 nm. In further embodiments, the averaged diameter is between about 400 and 500 nm.

In other embodiments, the averaged diameter is between about 50 and 500 nm. In other embodiments, the averaged diameter is between about 50 and 400 nm. In further embodiments, the averaged diameter is between about 50 and 300 nm. In further embodiments, the averaged diameter is between about 50 and 200 nm. In further embodiments, the averaged diameter is between about 50 and 100 nm. In further embodiments, the averaged diameter is between about 50 and 75 nm. In further embodiments, the averaged diameter is between about 50 and 60 nm.

The nanoparticles may each be substantially of the same shape and/or size. In some embodiments, the nanoparticles have a distribution of diameters such that no more than 0.01 percent to 10 percent of the particles have a diameter greater than 10 percent than the average diameter noted above, and in some embodiments, such that no more than 0.1, 0.2, 0.4, 0.6, 0.8, 1, 2, 3, 4, 5, 6, 7, 8, or 9 percent of the nanoparticles have a diameter greater than 10 percent than the average diameters noted above.

The PLGA polymer is a copolymer of polylactic acid (PLA) and polyglycolic acid (PGA), the copolymer being, in some embodiments, selected amongst block copolymer, random copolymer and grafted copolymer. In some embodiments, the copolymer is a random copolymer.

The PLGA is listed as Generally Recognized as Safe (GRAS) under Sections 201(s) and 409 of the Federal Food, Drug, and Cosmetic Act, and are approved for use in microparticulate systems.

In some embodiments, the nanoparticle is formed of a random copolymer of equimolar PLA and PGA, wherein the copolymer has a molecular weight of at least 4,500 Da, and is in the form of a nanoparticle having an averaged diameter between 100 and 200 nm.

In further embodiments, the nanoparticle is formed of a random copolymer of equimolar PLA and PGA, wherein the copolymer has a molecular weight of at least 4,500 Da, and is in the form of a nanoparticle having an averaged diameter between 50 and 100 nm.

In some embodiments, in the nanoparticles employed according to the invention, the PLA monomer is present in the PLGA in excess amounts.

In some embodiments, the molar ratio of PLA to PGA is selected amongst 95:5, 90:10, 85:15, 80:20, 75:25, 70:30, 65:35, 60:40, 55:45 and 50:50. In other embodiments, the PLA to PGA molar ratio is 50:50 (1:1).

Nanoparticles of the invention are used per se in formulations of the invention, to induce moisturizing/hydration of a skin portion to which the formulation is applied, to reduce skin dryness and other skin conditions which may or may not accompany certain medical conditions of the skin.

The nanoparticles may be used as such to induce at least one cosmetic effect or may be associated with at least one agent, which may be selected amongst cosmetically-active agents or non-active agents.

In some embodiments, the at least one agent is a cosmetically-active agent, capable of inducing, enhancing, arresting or diminishing at least one cosmetic non-systemic effect. The at least one cosmetically-active agent (substance, molecule, element, compound, entity, or a combination thereof) may be selected amongst dermatological agents, i.e., agents capable of inducing or modulating an effect on the skin of a subject, when administered in an effective amount, and non-active agents, i.e., which by themselves do not induce or modulate a dermatological effect but which may endow the nanoparticles with a selected characteristic, as will be further disclosed hereinbelow.

The at least one cosmetically-active agent may be selected amongst vitamins, proteins, anti-oxidants, peptides, polypeptides, lipids, carbohydrates, hormones, and other prophylactic agents, nutraceutical agents, small molecules (of a molecular weight of less than about 1,000 Da or less than about 500 Da), electrolytes, drugs, and any combination of any of the aforementioned. Non-limiting examples of such dermatologically-active agents may be vitamin c, ascorbyl palmitate, vitamin E, Zinc Picolinate, casein, oat protein, green tea extracts, grape seed extract, Pine Bark Extract, α-Lipoic acid, Coenzyme Q10, sage oil, Primrose Oil, Borage Oil, cysteine, proline, arginine/lysine polypeptide, caramel, and others.

In some embodiments, the at least one agent is a macromolecule (molecular weight above 1000 Da), which delivery through the skin layers is otherwise not possible. Such macromolecules may be hydrophilic (being partially or completely water soluble) or lipophilic (being hydrophobic-water insoluble).

In some embodiments, the at least one cosmetically-active agent is selected from hyaluronic acid, collagen, DHEA, and others. DHEA is meant to encompass also the sulphate derivatives thereof.

For certain applications, the at least one cosmetic agent is selected in accordance with its molecular weight. Thus, in some embodiments, the at least one cosmetic agent is selected to have a molecular weight higher than 1,000 Da. In other embodiments, the agent is selected to have a molecular weight of no more than 300 Da. In further embodiments, the dermatological agent is selected to have a molecular weight of between 500 and 1,000 Da.

The non-active agents which may be associated with the nanoparticles are selected to modulate at least one characteristic of the nanoparticle, such characteristic may for example be one or more of size, polarity, hydrophobicity/hydrophilicity, electrical charge, reactivity, chemical stability, clearance and targeting and others. In some embodiments, the non-active agent is a substantially linear carbon chain having at least 5 carbon atoms, and may or may not have one or more heteroatoms in the linear carbon chain.

In some embodiments, the non-active agent is selected from polyethylene glycols (PEG) of varying chain lengths, fatty acids, amino acids, aliphatic or non-aliphatic molecules, aliphatic thiols, aliphatic amines, and others. The agents may or may not be charged.

In some embodiments, the nanoparticle may be non-PEGylated, i.e., the non-active agent is different from PEG.

In some embodiments, the non-active agent is a fatty amino acid (alkyl amino acid), wherein optionally the alkyl portion of said alkyl amino acid has between 10 and 30 carbon atoms and may be linear or branched, saturated, semi saturated or unsaturated. In some embodiments, the amino acid portion of said alkyl amino acid may be selected amongst natural or non-natural amino acids, and/or amongst alpha- and/or beta-amino acids.

Depending on various parameters associated with the at least one cosmetically-active agent (the parameters being, for example, solubility, molecular weight, polarity, hydrophobicity/hydrophilicity, electrical charge, reactivity, chemical stability, biological activity, and others), or the at least one non-active agent, the agent may be contained (encapsulated) in said nanoparticle, embedded in the polymer matrix making up the nanoparticle and/or chemically or physically associated with the surface (whole surface or a portion thereof) of the nanoparticle. For the chosen application, the nanoparticle may therefore be in the form of core/shell (termed hereinafter also as nanocapsule), having a polymeric shell and a core which may be empty of any such agent or contain at least one agent. Alternatively the nanoparticles are of a substantially uniform composition, not featuring a distinct core/shell structure. These nanoparticles are herein referred to as nanospheres (NSs).

In some embodiments, the nanoparticles are devoid of the at least one agent, namely, the at least one agent resides on the surface of the nanoparticles.

Where nanocapsules are employed, the at least one (active or non-active) agent may be contained within the nanoparticles core (cavity), e.g., in an oily matrix, surrounded by a shell of the copolymer of the invention. In some embodiments, the shell comprises or is associated with the same or different agent.

In some embodiments, the nanoparticles are nanocapsules (NCs) containing at least one hydrophobic agent (the agent being contained in oil core and thus is lipophilic). Depending on a particular intended application, the oily core may be selected amongst any oily organic solvent or medium (single material or mixture), such materials may be selected, in a non-limiting fashion, from octanoic acid, oleic acid, glyceryl tributyrate, long chain triglycerides (such as soybean) and others.

Alternatively, relatively uniform structures, e.g., nanospheres (NSs) may be employed, where the at least one agent may be embedded within the nanoparticles matrix, e.g., homogenously, resulting in a nanoparticle in which the concentration of the agent within the nanoparticle is uniform.

In some embodiments, modification of the nanoparticles (either nanocapusles or nanospheres) surface may be required to enhance the effectiveness of the nanoparticles in the delivery of a cosmetically-active agent. For example, the surface charge of the nanoparticles may be modified to achieve modified biodegradation and clearance of the nanoparticles. The porosity of the polymer element of the particle (whether the core in the nanocapsule or the uniform matrix in the nanosphere) may also be optimized to achieve extended and controlled release of the cosmetically-active agent.

In another manifestation of the invention, the nanoparticles are modified to permit association with at least one (therapeutic or non-therapeutic) agent; the association may be a chemical association, such as a covalent bond, or a non-covalent bond such as an electrostatic bond, an ionic interaction, a dipole-dipole interaction, a hydrophilic interaction, van der Waal's force, a hydrogen bond, or a physical association of at least a portion of the agent with the nanoparticle. The physical association may be such that at least a portion of the at least one agent (or a linker moiety associated therewith) is entrapped, embedded, adsorbed or anchored into the nanoparticle element or surface. In one embodiment, the physical association occurs at the time of nanoparticle formation. Herein, the physical association is referred to in general as “physical anchoring”.

A nanoparticle may be associated with one or more of a variety of agents. For example, when two or more agents are used, they can be similar or different. Utilization of a plurality of agents in a particular nanoparticle can allow the targeting of multiple biological targets or can increase the affinity for a particular target. In addition, the nanoparticle may contain two agents, each having a different solubility-one hydrophobic (e.g., in the core) and one hydrophilic (e.g., in the shell or extending out of the particle).

The association between the nanoparticles and the agents may be selected, based on the intended application, to be labile, namely undergo dissociation under specific conditions, or non-labile. Typically, where the at least one agent is a cosmetically-active agent, it is either associated with the surface of the nanoparticles via labile bond(s) or via one or more linker moieties.

In some embodiments, the at least one agent is a cosmetically-active agent which association with the nanoparticles is via one or more linker moieties, the linker moiety being bifunctional, namely having a first (e.g., hydrophobic) portion which is capable of association (interaction) with the surface of the nanoparticles, and a second (e.g., hydrophilic) portion which is capable of association with the cosmetically-active agent.

The nanoparticle associated with one or a plurality of such linker moieties is referred to herein as a “modified nanoparticle”, namely a nanoparticle, as defined, which at least a part of its surface is associated with linker moieties which are chemically or physically capable of undergoing association with at least one agent. The plurality of linkers interacting with the surface of the nanoparticles, need not all be associated with cosmetic agents. Some may be associated with other non-active agents; others may have bare end-groups (unassociated with any agent). In some embodiments, the linkers are associated with one or more different cosmetic agents.

The association between the linker and the nanoparticle surface is typically selected from covalent bonding, electrostatic bonding, hydrogen bonding and physical anchoring (non-covalent) of at least a portion of the linker into the nanoparticle surface. The association between the linker and the at least one cosmetic agent is selected from covalent bonding, electrostatic bonding, and hydrogen bonding.

In some embodiments, the linker moiety is associated with one or both of (a) the at least one cosmetic agent and (b) the nanoparticle surface via covalent bonding. In other embodiments, the association between the linker and the nanoparticle surface is via anchoring, e.g., in the surface of the nanoparticle and may penetrate into the solid/oil core of the nanoparticle, of at least a portion of the linker into the nanoparticle surface, with another portion of the linker being exposed away from the nanoparticle surface.

In further embodiments, the linker is covalently bonded to said at least one cosmetically-active agent. In some embodiments, one or both of the following associations is labile: (a) the association of the linker with the cosmetically-active agent and (b) the association with the linker with the nanoparticle surface.

In some embodiments, in the nanoparticle having anchored (non-covalently) on its surface a plurality of linker moieties, each of said plurality of linker moieties is covalently bonded to at least one agent; both surface anchoring and covalent boding are labile.

The association of the linker and any of the nanoparticles and the agent may be labile, namely the linker may be a readily cleavable linker, which is susceptible to dissociation under conditions found in vivo. For example, where the nanoparticles of the invention are employed as delivery systems for cosmetic skin applications, upon passing into and through one or more skin layers, the cosmetically-active agent may be released from the linker or the nanoparticles carrier. Readily cleavable associations can be such that are cleaved by an enzyme of a specific activity or by hydrolysis. For skin applications, the association between the linker and the cosmetically-active agent or between the nanoparticles and the linker may be selected to be cleavable by an enzyme present in one or more layers of skin tissue.

In some embodiments, the linker moiety contains a carboxylic acid group (to form esters) or a thiol group (to form a sulfide bond).

In other embodiments, the linker moiety is selected according to the half-life of the cleavable association, namely the quantity of the cosmetically-active agent that becomes dissociated from the linker. In some embodiments, the association of the linker to the cosmetically-active agent has a half-life of between 1 minute and 48 hours. In some embodiments, the half-life is below 24 hours.

In further embodiments, the linker moiety comprises a functional group selected from —S—, —NH—, —C(═O)O—, —C(═O)S—, —C(═O)NH—, —C(═S)NH—, —OC(═O)NH—, —NH(═O)NH—, —S(═O)NH—, —S(═O)₂NH—, and others.

In some embodiments, the linker is selected amongst polyethylene glycols (PEG) of varying chain lengths.

In some embodiments, the linker moiety is a fatty amino acid (alkyl amino acids), wherein the alkyl portion has between 10 and 30 carbon atoms and may be linear or branched, saturated, semi saturated or unsaturated. The amino acid portion may be selected amongst natural or non-natural amino acids, and/or amongst alpha- and/or beta-amino acids. The amino acid group of the linker may be derivable from an amino acid selected, without limitation, from alpha and beta amino acids.

In some embodiments, the linker is a fatty cysteine having an alkyl chain of at least 10 carbon atoms.

In further embodiments, the linker is oleylcysteineamide of the formula I:

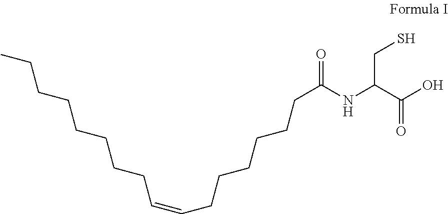 [SMILES: CCCCCCCC/C=C\CCCCCCC(=O)NC(CS)C(=O)O]

In some embodiments, the linker moiety is a thiolated compound, and thus the modified nanoparticle is a thiolated nanoparticle capable of association with, e.g., cosmetically-active macromolecules (molecular weight above 1000 Dalton), cosmetically-active hydrophilic molecules and electrolytes. The association between the thiolated nanoparticle and the agent may be via an active group on the agent, such group may be a maleimide functional group.

In some embodiments, the at least one agent is associated with the surface of the nanoparticle and at least one another agent is associated to be contained within a core of said nanoparticle or within a matrix of said nanoparticle, i.e. the at least one agent may be hydrophilic, while the at least one another agent may be lipophilic. In further embodiments, the at least one agent is associated with the surface of the nanoparticle via one or more linker moieties such as those described herein, namely linker moieties having a first portion capable of association with the nanoparticle and a second portion capable of association with the cosmetically-active agent.

The present invention also provides a polymeric nanoparticle having on its surface a plurality of cosmetically-active agents, each agent being associated (bonded) to said nanoparticle via a linker moiety, the nanoparticles being of a polymeric material selected from poly(lactic acid) (PLA), poly(lacto-co-glycolide) (PLG), poly(lactic glycolic) acid (PLGA), poly(lactide), polyglycolic acid (PGA), poly(caprolactone), poly(hydroxybutyrate) and/or copolymers thereof. In some embodiments, said polymeric material is selected from PLA, PGA and PLGA. In further embodiments, the polymeric nanoparticles are of PLGA.

In some embodiments, the linker moiety is oleylcysteineamide. In other embodiments, the nanoparticle has the physical characteristics disclosed hereinabove. In some embodiments, the nanoparticle is a poly(lactic glycolic) acid (PLGA) nanoparticle having an averaged diameter of at most 500 nm, the PLGA having an averaged molecular weight of up to 20,000 Da.

The nanoparticles employed in the compositions of the invention may be formulated into cosmetic formulations or may be used in methods of cosmetic applications. The concentration of nanoparticles in a cosmetic composition according to the invention may be selected so that the amount is sufficient to deliver a desired effective amount of a cosmetically-active agent to the subject. As known, the “effective amount” for purposes herein may be determined by such considerations as known in the art. The amount must be effective to achieve the desired dermatological effect, e.g., promote the normalization of the cell function, without substantially inducing a systemic effect. The effective amount is determined depending, inter alia, on the type and severity of the skin condition to be treated and the treatment regime. The effective amount is typically determined in appropriately designed clinical trials (dose range studies) and the person versed in the art will know how to properly conduct such trials in order to determine the effective amount.

As used herein, the terms “dermatologic” and “cosmetic” are used interchangeably to denote application of a composition or formulation according to the invention onto a skin region of a subject. The application, as disclosed herein, is intended to treat or prevent a skin condition and/or improve the physical appearance of the skin region.

The compositions of the invention may be used as skin moisturizing/hydration agents. Alternatively, the formulations may be used for treating a topical, non-systemic, condition associated with a skin condition selected from atopic and contact dermatitis, psoriasis, eczema, thyroid disorders, ichtyosis, scleroderma and Sjorgen's disease, infection skin diseases caused by microorganisms, such as fungi, microbes, inflammatory or allergy; acne, hives (urticarial), pigmentation, stings, and bites, pruritic conditions, or alopecia.

The cosmetic composition of the invention may comprise varying nanoparticle types or sizes, of different or same dispersion properties, utilizing different or same dispersing materials.

The nanoparticles may also be used as delivery systems to transport a wide range of cosmetically-active agents topically. The nanoparticle delivery systems of the invention facilitate targeted delivery and controlled release applications, enhance bioavailability at the site of action also reduce dosing frequency, and minimize side effects.

In most general terms, the delivery system of the invention comprises:
(i) a polymeric nanoparticle, as disclosed herein; and
(ii) at least one agent associated with said nanoparticle, said at least one agent being optionally associated with the nanoparticle surface via a linker moiety having.

Without wishing to be bound to theory, small active agents which are hydrophilic (such as amino acids and electrolytes) do not usually penetrate the skin. Therefore, conjugation of such agents to nanoparticles in accordance with the invention significantly enhances the skin penetration of the agents, and may prolong their release into the skin.

Hydrophilic and lipophilic agents, i.e., macromolecules, cannot penetrate or diffuse through the skin due to their high molecular weight. Such macromolecules may be linked to the surface of the nanoparticles, thereby enhancing and/or prolonging their release into the skin.

In addition, small molecules which penetrate the skin under normal conditions however are not retained therein over time, may be entrapped in the nanoparticles of the invention, affording prolonged release into the skin and minimizing their diffusion within blood circulation (as is the case of steroids, for example).

In some embodiments, the linker has a first portion physically anchored (non-covalently associated) to said surface and a second portion associated with said at least one cosmetically-active agent. In some embodiments, the first portion physically anchored to said surface is hydrophobic, and the second portion associated with said at least one agent is hydrophilic.

The delivery system of the invention is capable of delivering the cosmetically-active agent at a rate allowing controlled release of the agent over at least about 12 hours, or in some embodiments, at least about 24 hours, or in other embodiments, over a period of 10-20 days.

The cosmetic composition of the invention comprises a cosmetically acceptable carrier. In some embodiments, the cosmetically acceptable carrier is a silicone-based carrier. In further embodiments, the silicon based carrier is anhydrous, thereby providing a composition essentially free of water.

The cosmetic composition of the invention may be formulated as emulsions, creams, lotions, gels, ointments, skin protective creams or skin protective ointments, sprays, aerosols, sticks, decorative cosmetic formulations, powders, disinfectants, skin tonics, skin cleansing products, skin peeling formulations, suspensions, soaps, bathing additives such as bathing gels, mouth wash, tooth paste, chewing gum, shampoos, sunscreen products, UV protection products, medical bandages, medical plasters, wound dressings, tampons, diapers, formulations for applying to baby-soothers, formulations for vaginal application, and antiseptic fluid formulations for rinsing/irrigation of body cavities.

In some embodiments, the compositions are formulated as water-free or dry formulation, namely as formulations essentially free of water. Thus, the invention also provides essentially-water free formulations for topical applications, wherein said formulations comprising a cosmetic composition according to the present invention and any of the embodiments recited herein.

The delivery systems are typically topically administered as cosmetic formulations, comprising the system and a cosmetically acceptable carrier. The cosmetically acceptable carrier may be selected from vehicles, adjuvants, excipients, and diluents, which are readily available to the public. The cosmetically acceptable carrier is selected to be chemically inert to the delivery system of the invention or to any component thereof and one which has no detrimental side effects or toxicity under the conditions of use.

The choice of carrier will be determined in part by the particular cosmetically-active agent. The cosmetic compositions or the delivery system of the present invention are formulated for topical, transepithelial, epidermal, transdermal, and/or dermal administration routes.

The delivery system can be administered in a biocompatible aqueous or lipid solution. This solution can be comprised of, but not limited to, saline, water or a cosmetically acceptable organic medium. The delivery system of the invention may also be topically administered as a dry formulation, namely a delivery system essentially free of water.

The administration of a delivery system formulation can be carried out at a single dose or at a dose repeated once or several times after a certain time interval. The appropriate dosage may vary according to such parameters as the cosmetically effective dosage as dictated by and directly dependent on the individual being treated, the unique characteristics of the cosmetically-active agent and the particular cosmetic effect to be achieved. Appropriate doses can be established by the person skilled in the art.

The cosmetic composition of the present invention may be selected to improve or prevent at least one condition of a skin region. The term “treatment” or any lingual variation thereof, as used herein, refers to the administering of a cosmetically effective amount of a cosmetic composition of the present invention which is effective to improve or prevent a skin condition (disorder), without inducing a systemic effect.

As known, human skin is made of numerous layers which may be divided into three main group layers: Stratum corneum which is located on the outer surface of the skin, the epidermis and the dermis. While the Stratum corneum is a keratin-filled layer of cells in an extracellular lipid-rich matrix, which in fact is the main barrier to drug delivery into skin, the epidermis and the dermis layers are viable tissues. While transdermal delivery of drugs seems to be the route of choice, only a limited number of agents can be administered through this route. The inability to transdermally deliver a greater variety of drugs depends mostly on the requirement for low molecular weight (drugs of molecular weights not higher than 500 Da), lipophilicity and small doses of the drug.

The delivery system of the invention clearly overcomes these obstacles. As noted above, the system of the invention is able of holding cosmetically-active agents of a great variety of molecular weights and hydrophilicities. The delivery system of the invention permits the transport of the at least one cosmetically-active agent across at least one of the skin layers, across the Stratum corneum, the epidermis and the dermis layers. Without wishing to be bound by theory, the ability of the delivery system to transport the cosmetically-active agent across the Stratum corneum depends on a series of events that include diffusion of the intact system or the dissociated cosmetically-active agent and/or the dissociated nanoparticles through a hydrated keratin layer and into the deeper skin layers.

The cosmetic composition of the invention may be anhydrous or non-anhydrous formulations. In some embodiments, the formulations are anhydrous, namely dry-formulations.

In some embodiments, the formulation according to the present invention comprises at least one nanoparticle of a polymer selected from PLA, PGA and PLGA and one or more of the following ingredients: dimethicone crosspolymer (Dow corning 9040); dimethicone; cyclopentasiloxane; Shin etsu KSG-16 dimethicone; boron nitride; lauroyl lysine Ajinomoto; hyaluronic acid MP 50000; palmitoyloligopeptide-biopeptide CL Sederma and palmitoyl tetrapeptide-N-palmitoyl-rigin.

In further embodiments, the formulation comprises ingredients according to Table 1.

TABLE 1
ingredients of a formulation according to the invention

| Ingredient | Relative amount/100 |
|---|---|
| Dow corning 9040 - | 40.0-50.0 |
| Cyclopentasiloxane (and) | |
| Dimethicone crosspolymer | |
| Dimethicone | 5.0-7.0 |
| Cyclopentasiloxane | 10.0-15.0 |
| Shin etsu KSG-16 | 20.0-35.0 |
| Dimethicone (and) | |
| Dimethicone/Vinyl | |
| dimethicone crosspolymer | |
| Boron Nitride | 0.3-0.70 |
| lauroyl Lysine Ajinomoto | 0.2-0.70 |
| hyaluronic acid MP 50000 | 0.1-0.40 |
| Palmitoyloligopeptide - | 0.05-0.3 |
| Biopeptide CL Sederma | |
| Palmitoyl tetrapeptide - N- | 0.05-0.3 |
| Palmitoyl-Rigin | |

The invention also provides a delivery system for topical application which comprises:
(i) a polymeric nanoparticle as disclosed herein; and
(ii) at least one agent (cosmetically-active or non-active agent) associated with said nanoparticle, said at least one agent being optionally associated with the nanoparticle surface via a linker moiety.

In some embodiments, the linker is oleylcysteineamide.

The invention also provides a multistage delivery system which comprises:
(i) a polymeric nanoparticle as disclosed herein;
(ii) a linker moiety associated with the surface of said polymeric nanoparticles;
(iii) at least one cosmetically-active agent associated with linker moiety; and
(iv) optionally at least one additional agent which may be associated with the nanoparticle.

With the ability of the delivery system of the invention to dissociate under biological conditions, the multistage system provides one or more of the following advantages: (1) the multistage system permits the transport of the cosmetically-active agent through a tissue barrier by various mechanisms; (2) the cosmetically-active agent may be dissociated from the linker or from the nanoparticle (in cases where the agent is directly associated with the nanoparticle) and thus is deliverable to a particular target tissue or organ in the body of a subject administered with the delivery system; (3) the modified nanoparticle, which comprises the polymeric nanoparticle and the linker moiety (free of the cosmetically-active agent), may further travel through additional barrier tissues, increasing their hydration and inducing additional cosmetic effects; and (4) where the nanoparticles are nanocapsules also holding an agent within the capsule core, they may allow for simultaneous delivery and localization of a plurality of cosmetically-active agents.

Accordingly, in the delivery system of the invention, each component may be designed to have a separate intended function, which may be different from an intended function of another component. For example, the cosmetically-active agent may be designed to target a specific site, which may be different from a site targeted by the linker moiety or the bare nanoparticle, and thus overcome or bypass a specific biological barrier, which may be different from the biological barrier being overcome or bypassed the system as a whole. For example, the incorporated cosmetically-active agent can be mostly released from the nanoparticles while the nanoparticle can be fragmented or biodegraded more slowly and be eliminated through the dermis as monomers of PLA or PGA.

In another non-limiting example, the delivery system may be designed to include clearance resistant agents. While elimination of nanoparticles by macrophages is less common in topical administration, addition of agents, such as PEG, reduces clearance by the tissue, thereby improving and/or prolonging the stability of the nanoparticles in the tissue post-application.

The invention also provides a process for the preparation of a delivery system according to the invention, the process comprising:
obtaining a nanoparticle, as defined herein;
reacting said nanoparticle with a linker moiety under conditions permitting association between the nanoparticle surface and the linker moiety, to thereby obtain a surface-modified nanoparticle; and
contacting the surface modified nanoparticle with at least one cosmetically-active agent, to allow association between the linker end group; to thereby obtain a delivery system in accordance with the present invention.

In some embodiments, the linker moiety may be associated with the cosmetically-active agent prior to the contacting with the nanoparticle and the process may thus comprise:
obtaining a nanoparticle, as define herein;
obtaining a cosmetically-active agent associated linker moiety; and
reacting the cosmetically-active agent associated linker with said nanoparticle to permit association of at least a portion of said linker with the surface of the nanoparticle.

In some embodiments, the delivery system/multistage system comprises nanoparticles associated with oleylcysteineamide, which is anchored at the interface of nanoparticles and thus may be easily applied to a PLGA polymer of different molecular weights, resulting in a wide range of thiolated nanoparticles.

The linking process does not require a priori chemical modification of the particle-forming polymer. This is achieved by the use of a molecular linker, e.g., oleylcysteineamide, having a lipophilic portion which non-covalently anchors to the particle's polymeric matrix or polymeric nanocapsule wall and a second portion comprising a thiol compound to which it is possible, in a subsequent step, to bind the desired cosmetically-active agent either directly or activated by a maleimide group. This approach eliminates the need to tailor for each different cosmetically-active agent a different nanoparticle composition, and enables a generic linker, which can be used for different cosmetic applications.

Other than employing the methods available for chemically associating the cosmetically-active agent to the linker, e.g., carbodimide mediated conjugation, the thiol modified nanoparticle surface may be used also or alternatively for the chelation and dermal delivery of vital electrolytes, e.g., divalent metals, such as copper, selenium, calcium, magnesium and zinc. The thiolated nanoparticles may also serve as a delivery system to chelate undesired excess amounts of metals and thus reduce the metal catalyzed ROS (Reactive Oxygen Species) mediated deleterious effect on the skin.

Also provided are polylactic acid (PLA) nanoparticles having an averaged diameter of at most 500 nm, the PLA having an averaged molecular weight of up to 10,000 Da.

In some embodiments, the PLA has an averaged molecular weight of between 1,000 and 10,000 Da. In other embodiments, the PLA has an averaged molecular weight of between 1,000 and 5,000 Da. In further embodiments, the PLA has an averaged molecular weight of between 1,000 and 3,000 Da. In still other embodiments, the PLA has an averaged molecular weight of about 1,000, about 2,000, about 3,000, about 4,000 or about 5,000 Da.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A-B are CRYO-TEM images of blank PLGA₄₅₀₀ nanoparticles at various areas of the carbon grid (FIG. 1A) and blank PLGA₄₅₀₀ nanoparticles at various areas of the carbon grid following one month storage at 4° C. (FIG. 1B).

FIGS. 2A-B are CRYO-TEM images of DHEA loaded PLGA₄₅₀₀ nanocapsules at various areas of the carbon grid (FIG. 2A) and DHEA loaded PLGA₅₀₀₀₀ nanocapsules at various areas of the carbon grid (FIG. 2B).

FIG. 3 is a collection of fluorescent images of various consecutive tape-stripping following topical administration over 3 h of different NIR-PLGA nanosphere formulations (2.25 mg/cm²). Scanning was performed using ODYSSEY® Infra Red Imaging System.

FIGS. 4A-D is a depiction of reconstructed fluorescent images of whole skin specimens, 2 h following topical administration of DiD incorporated nanocapsules or nanospheres (4.5 mg/cm²). FIG. 4A-DiD loaded PLGA₄₅₀₀ nanospheres; FIG. 4B-DiD loaded PLGA₅₀₀₀₀ nanospheres; FIG. 4C-DiD control solution; FIG. 4D-DiD loaded PLGA₄₅₀₀ nanocapsules. Z stack scanning was performed using a Zeiss LSM 710 confocal microscope.

FIGS. 5A-E is a depiction of reconstructed fluorescent images of whole skin specimens, 2 h following topical administration of varied fluorescent nanocapsules or nanospheres (3.75 mg/cm²). FIG. 5A-DiD incorporated and rhodamine B conjugated PLGA₄₅₀₀ nanospheres; FIG. 5B-DiD incorporated and rhodamine B conjugated PLGA₄₅₀₀ nanocapsules; FIG. 5C-Rhodamin B incorporated latex nanospheres; FIG. 5D-DiD and rhodamine B conjugated PLGA₄₅₀₀ aqueous dispersion control; FIG. 5E-DiD and rhodamine B conjugated PLGA₄₅₀₀ MCT containing aqueous dispersion control. Z stack scanning was performed using a Zeiss LSM 710 confocal microscope.

FIGS. 6A-B exhibits DiD (FIG. 6A) and Rhodamine B (FIG. 6B) cumulative fluorescence intensity as a function of skin depth following 2 hours topical administration of various DiD incorporated RhdB-PLGA formulations (3.75 mg/cm²) using 27 μm incremental optical sectioning.

FIGS. 7A-D CLSM images of 8 μm thick vertical skin sections 2 h after topical administration of DID incorporated RhdB-PLGA NPs (FIG. 7A) and NCs (FIG. 7B) and their respective controls (FIG. 7C and FIG. 7D) (3.75 mg/cm²). Bar=100 μm.

FIG. 8 exhibits Rhodamine B cumulative fluorescence intensity as a function of skin depth following 2 hours topical administration of various rhodamine B incorporated formulations including PLGA nanospheres, nanocapsules and latex nanospheres (3.75 mg/cm²) using 27 μm incremental optical sectioning.

FIGS. 9A-D [³H]DHEA (FIG. 9A and FIG. 9C) and [³H]COE (FIG. 9B and FIG. 9D) distribution in the viable epidermis (FIG. 9A and FIG. 9B) and dermis (FIG. 9C and FIG. 9D) skin compartments over time following incubation of various radioactive nanocarriers and their respective controls. FIG. 9A and FIG. 9C: positively (♦) and negatively (▪) charged [³H]DHEA NCs and their respective oil controls (⋄, □); FIG. 9B and FIG. 9D: [³H]COE NSs (▴), [³H]COE NCs () and their respective controls (Δ, ◯). Significant difference (P value<0.05) of the positively (*) and negatively (**) charged DHEA NCs in comparison to their respective controls.

FIG. 10 exhibits [³H]DHEA amounts recorded in the receptor compartment fluids following topical application of positive (♦) and negative (▪) DHEA loaded NCs and their respective oily controls (⋄, □). Values are mean±SD. Significant difference (P value<0.05) of the positively (*) and negatively (**) charged DHEA NCs in comparison to their respective controls.

FIGS. 11A-D are CRYO-TEM images of PLGA₄₅₀₀ nanoparticles conjugated to hyaluronic acid (300 KDa), at various areas of the carbon grid. FIGS. 11A-B and 11C-D are from two different batches.

FIGS. 12A-D show TEM images of NPs dispersed in anhydrous silicone cream incubated at RT over 70 days.

FIGS. 13A-D show TEM images of NCs dispersed in anhydrous silicone cream incubated at RT over 70 days.

FIGS. 14A-D show TEM images of NPs dispersed in anhydrous silicone cream incubated at RT over 10 days following incubation at 37° C. over 60 days.

FIGS. 15A-D show TEM images of NCs dispersed in anhydrous silicone cream incubated at RT over 10 days following incubation at 37° C. over 60 days.

FIGS. 16A-D show TEM images of NSs dispersed in anhydrous silicone cream incubated at RT over 40 days following incubation at 60° C. over 30 days.

FIGS. 17A-D show TEM images of NCs dispersed in anhydrous silicone cream incubated at RT over 40 days following incubation at 60° C. over 30 days.

FIGS. 18A-D show TEM images of: (FIG. 18A) MCT NCs; (FIG. 18B) oleic acid NCs; (FIG. 18C) NPs; (FIG. 18D) HA NPs.

FIGS. 19A-D show TEM images using negative staining of 2% phosphotungstic acid pH 6.4 of: reconstituted powders of NPs prepared in large scale (FIGS. 19A-B); and NPs dispersed in anhydrous silicone cream (FIGS. 19C-D).

FIGS. 20A-D show TEM images using negative staining of 2% phosphotungstic acid pH 6.4 of: reconstituted powders of HA NPs prepared in large scale (FIGS. 20A-B); and HA NPs dispersed in anhydrous silicone cream (FIGS. 20C-D).

FIGS. 21A-D show TEM images using negative staining of 2% phosphotungstic acid pH 6.4 of: reconstituted powders of DHEA NCs prepared in large scale (FIGS. 21A-B); and DHEA NCs dispersed in anhydrous silicone cream (FIGS. 21C-D).

### DETAILED DESCRIPTION OF THE INVENTION

### I. Lactic Acid and Glycolic Delivery to the Skin

Use is made of the clinically well-accepted PLGA polymers as well as PLA particles of a specific molecular weight, to prepare nanoparticles of a certain particle size that are applied onto the skin, penetrate in the upper layers of the dermis and release, in a controlled manner over time, lactic and glycolic acid, or only lactic acid, which are natural moisturizing factors, allowing a prolonged and sustained hydration of the skin without being harmful.

The PLGA nanoparticles, per se, empty or loaded with appropriate actives, namely cosmetically-active agents, are used as the prolonged active hydrating ingredients, as a result of their degradation within the skin leading to the progressive and continuous release of lactic and glycolic acid. Even if the nanoparticles penetrate into the deep layer of the epidermis or even the dermis, they do not induce any damage as previously described since the hydrolysis product lactic and glycolic acids are naturally eliminated or excreted.

It should be emphasized the PLGA, as the active hydrating components of the composition of the invention, are not merely used as carriers for delivery of other components to the skin, although the invention also encompasses the possibility that other beneficial active components are used. Thus, in accordance with the invention the composition is intended for topical application, i.e., contains carriers for topical applications, as well as for other applications.

The nanoparticles of the invention are typically of a size smaller than 500 nm. Typically, the nanoparticles are of a size range of between 100 and 200 nm, or between 50 and 100 nm.

In some embodiments, the molecular weight of PLGA and the ratio between PLA and PGA is tailored so that the nanoparticles have the following properties:
(a) Penetrate into the skin to at least the 10 superficial epidermis layers;
(b) Penetrate to at least 4-20 micrometers into the skin;
(c) Biodegrade in the skin layer into which they penetrate (typically about 15% in the Stratum corneum);
(d) Sustained release of the lactic acid and glycolic acid or only the lactic acid for a period above 24 hours, preferably above 72 hours, more preferably about a week.

Without wishing to be bound by theory, there seems to be an interplay between the size of particle (which influences the penetration rate and the depth of penetration), the ratio of PLA and PGA and the molecular weight of the PLGA, in such a way that the above properties can be achieved by a number of combinations. Several changes in parameters may neutralize each other.

In some embodiments, the ratio of PLA:PGA is 85:15; 72:25; or 50:50. In some embodiments, the ratio is 50:50.

In other embodiments, the molecular weight of the PLGA ranges from 2,000 to 10,000 Da. In some embodiments, the ratio is between 2,000 and 4,000 Da.

In other embodiments, the PLA particles may be employed per se, in such embodiments the PLA molecular weight is in the range of 4,000 and 20,000 Da.

### II. Encapsulation Strategies of Insoluble Compounds in Nanoparticles-Cosmetic Applications of DHEA Loaded PLGA Nanoparticles

In the present invention, the nanoparticles may be loaded with cosmetically-active materials, as disclosed hereinabove.

Humans have adrenals that secrete large amounts of dehydroepiandrosterone (DHEA) and its sulphate derivatives (DHEAS). A remarkable feature of DHEA(S) plasma levels in humans is their great decrease with aging. Researchers have postulated that this age-related decline in DHEA(S) levels may explain some of the degenerative changes associated with aging. Three mechanisms of action of DHEA(S) have been identified. DHEA and DHEA(S) are precursors of testosterone and estradiol. DHEA(S) is a neurosteroid, which modulates neuronal excitability via specific interactions with neurotransmitter receptors, and DHEA is an activator of calcium-gated potassium channels.

Randomized, placebo-controlled clinical trials which included 280 healthy individuals (140 men and 140 women) aged 60-years and over treated with (near) physiological doses of DHEA (50 mg/day) over one year have yielded very positive results. Impact of DHEA replacement treatment was assessed on mood, well being, cognitive and sexual functions, bone mass, body composition, vascular risk factors, immune functions and skin. Interestingly, an improvement of the skin status was observed, particularly in women, in terms of hydration, epidermal thickness, sebum production, and skin pigmentation. Furthermore, no harmful consequences were observed following this 50 mg/day DHEA administration over one year.

It is also known that DHEA might be related to the process of skin aging through the regulation and degradation of extracellular matrix protein. It was demonstrated that DHEA can increase procollagen synthesis and inhibit collagen degradation by decreasing matrix metalloproteinase (MMP)-1 synthesis and increasing tissue inhibitor of matrix metalloprotease (TIMP-1) production in cultured dermal fibroblasts. DHEA (5%) in ethanol:olive oil (1:2) was topically applied to buttock skin of volunteers 12 times over 4 weeks, and was found to significantly increase the expression of procollagen alpha1 (I) mRNA and protein in both aged and young skin. On the other hand, topical DHEA significantly decreased the basal expression of MMP-1 mRNA and protein, but increased the expression of TIMP-1 protein in aged skin. These recent results suggest the possibility of using DHEA as an anti-skin aging agent.

Based on the overall reported results, exogenous DHEA administered topically may promote keratinization of the epidermis, enhance skin hydration by increasing the endogenous production and secretion of sebum subsequently reinforcing the barrier effect of the skin, treat the atrophy of the dermis by inhibiting the loss of collagen and connective tissue and finally can modulate the pigmentation of the skin. These properties render DHEA the active of choice as an anti-aging active ingredient provided DHEA is adequately dissolved in the topical formulation, can diffuse from the formulation towards the skin and be fully bioavailable for skin penetration following dermal application. Indeed, DHEA exhibits complex solubility limitations in common cosmetic and pharmaceutical solvents such as water, polar oils and vegetable oils. DHEA is practically insoluble in water (0.02 mg/ml) and is known for its tendency to precipitate rapidly within topical regular formulations even at concentrations lower than 0.5%, yielding several polymorphic crystal forms which are difficult to control and exhibit very slow dissolution rate.

Furthermore, DHEA shows low solubility in lipophilic phases with a maximum solubility of 1.77% in mid chain triglycerides (MCT). The most accepted topical dosage form is the o/w emulsion in which the DHEA should be dissolved in the lipophilic phase. However, this solution is very difficult to accomplish since very high concentrations of oil phase (more than 70%) may be needed to achieve a DHEA concentration eliciting an adequate efficacy activity (approximately 0.5% w/v). Topical products with such high oil phases will not be pleasant and will not meet definitely the appealing cosmetic requirements. There is no doubt that the recrystallization process of DHEA should be prevented since it can potentially cause significant variations in therapeutic bioavailability and efficacy. The drug crystals need first to re-dissolve in the skin prior to diffuse and penetrate the superficial skin layers. Such a process is unlikely to occur easily and will affect significantly the activity of the product. Moreover, the recrystallization process can affect the stability and the physical appearance of the formulation. Thus, there is clearly a need to prepare pleasant and convenient o/w topical formulations where DHEA loaded nanoparticles can be dispersed at an adequate concentration and will not exhibit any precipitation process. Furthermore, the DHEA embedded nanocarrier should be incorporated in a topical formulation, which can promote penetration of the active ingredient within the epidermis and dermis layers where its action is most needed.

III. Delivery of Surface Bound Cosmetically-Active Macromolecules and Minerals into the Skin Using Thiol Activated Nanoparticles

Commercially available products utilizing transdermal delivery have been mainly limited to low molecular weight lipophilic drugs (MW<500 Da) [17], with larger molecular weights (MW>500 Da) facing penetration difficulties [18]. Due to the impervious nature of the stratum corneum towards macromolecules, a suitable penetration enhancer should substantially improve transport of macromolecules through the skin. Various technologies have been developed for this purpose, including the use of microneedles, electroporation, laser generated pressure waves, hyperthermia, low-frequency sonophoresis, iontophoresis, penetration enhancers, or a combination of these methods. Many penetration enhancement techniques face inherent challenges, such as scale-up and safety concerns [18]. The present invention proposes the delivery of cosmetically-active macromolecules, hydrophilic and lipophilic, by a non-invasive method, using a surface binding technique of macromolecules to thiolated nanoparticles or encapsulation technique.

### Thiolated NPs—State of the Art

Nanoparticles can be functionalized with a maleimide moiety, which is then conjugated to a thiolated protein. Alternatively, nanoparticles can be functionalized with a thiol group then conjugated to a maleimidic residue on the protein. Traditionally, such delivery systems have been mostly used for the targeted delivery of drug loaded nanoparticles, principally to malignant tumors, where the surface conjugated protein is used simply as a targeting moiety recognizing disease specific epitopes.

### IV. Experimental

1. DiD Loaded PLGA NPs and NCs and/or Rhodamine B PLGA Conjugated NPs or NCS Preparation:

PLGA was dissolved in acetone containing 0.2% w/v Tween 80, at a concentration of 0.6% w/v. In case where NCs were prepared, Octanoic acid or MCT (medium chain triglyceride) at a concentration of 0.13% w/v was also added to the organic phase. If DiD loaded NPs were prepared, then an aliquot of DiD in acetone solution at a concentration of 1 mg/ml was also added to the organic phase, resulting in a final concentration of 15-30 μg/ml. If rhodamine B PLGA conjugated NPs or NCs were prepared, 0.03% w/v rhodamine B tagged PLGA was dissolved in acetone together with 0.57% w/v non-labeled PLGA. The organic phase was added to the aqueous phase containing 0.1% w/v Solutol® HS 15. The suspension was stirred at 900 rpm for 15 minutes and then concentrated by evaporation to a final polymer concentration of 30 mg/ml. The aqueous and oil control composition was identical to the formulation described above, only without the polymer presence.

### 2. [³H]DHEA and [³H]COE PLGA Solid Nanoparticle Encapsulation and Evaluation in Cosmetic Applications

### DHEA NPs Preparation

DHEA loaded PLGA nanocapsules were prepared using the interfacial deposition method [19]. DHEA was solubilized in octanoic acid/MCT/oleic acid and in acetone. If positively charged DHEA NCs were prepared, the cationic lipid, DOTAP [1,2-dioleoyl-3-trimethylammonium-propane], at a concentration of 0.1% w/v was added to the organic phase. In case radioactive-labeled DHEA NCs were prepared, 15 μCi of tritiated DHEA were inserted into the oil core of the NCs during their preparation, together with 1 mg of cold DHEA. In case [³H]Cholesteryl oleyl ether ([³H]COE) were prepared, 80 and 127 μCi [³H]COE were either, dissolved in MCT to form NCs, or simply added to the organic phase for NPs formation, respectively. The organic phase was added drop wise to the aqueous phase under stirring at 900 rpm, and the formulation was concentrated by evaporation to a polymer concentration of 8 mg/ml. The formulations were filtered through 0.8 μm membrane and then 3 ml from the different [³H]DHEA NCs were dia-filtrated with 30 ml PBS (pH 7.4) (Vivaspin 300,000 MWCO, Vivascience, Stonehouse, UK) and filtered through 1.2 μm filter (w/0.8 μm Supor® Membrane, Pall corporation, Ann Arbor, USA). The radioactivity intensity for the overall formulations and their respective controls was set, such that a finite dose applied was in the range of a total of 0.63-1.08 μCi/ml. The compositions of the organic phase and the aqueous phase are presented in Table 2.

TABLE 2
compositions of organic phase and aqueous phase

| Organic phase | Aqueous phase |
|---|---|
| PLGA 4500 MW - 150 mg | Solutol HS 15-50 mg |
| Octanoic acid - 75 μl | Water - 100 ml |
| DHEA - 10 mg | |
| TWEEN 80-50 mg | |
| Acetone - 50 ml | |

Particle size analysis: mean diameter and particle size distribution measurements were carried out utilizing an ALV Noninvasive Back Scattering High Performance Particle Sizer (ALV-NIBS HPPS, Langen, Germany) at 25° C. and using water as diluent.

Zeta potential measurements: the zeta potential of the NPs was measured using the Malvern zetasizer (Malvern, UK) diluted in HPLC grade water.

Scanning (SEM) and Transmission electron microscopy (TEM): morphological evaluation was performed by means of scanning and transmission TEM (Philips Technai F20 100 KV). Specimens for TEM visualization are prepared by mixing the sample with phosphotungstic acid 2% (w/v) pH 6.4 for negative staining.

Cryo-Transmission Electron Microscopy (Cryo-TEM):

A drop of the aqueous phase was placed on a carbon-coated holey polymer film supported on a 300 mesh Cu grid (Ted Pella Ltd), the excess liquid was blotted and the specimen was vitrified via a fast quench in liquid ethane to −170° C. The procedure was performed automatically in the Vitrobot (FEI). The vitrified specimens were transferred into liquid nitrogen for storage. Such fast cooling is known to preserve the structures present at the bulk solution and therefore provides direct information on the morphology and aggregation state of the objects in the bulk solution without drying. The samples were studied using a FEI Tecnai 12 G2 TEM, at 120 kV, with a Gatan cryo-holder maintained at −180° C., and images were recorded on a slow scan cooled charge-coupled device CCD camera Gatan manufacturer. Images were recorded with the Digital Micrograph software package, at low dose conditions, to minimize electron beam radiation damage.

### 3. Diffusion Experiments

Franz diffusion cells (Crown Glass, Sommerville, N.J., USA) with an effective diffusion area of 1/0.2 cm² and an acceptor compartment of 8 ml were used. The receptor fluid was a phosphate buffer, pH 7.4.

Throughout the experiment, the receptor chamber content was continuously agitated by a small magnetic stirrer. The temperature of the skin was maintained at 32° C. by water circulating system regulated at 37° C. Finite doses of the vehicle and formulations (10-50 mg polymer per cell) were applied on the horny layer of the skin or cellulose membrane. The donor chamber was opened to the atmosphere. The exact time of application was noted and considered as time zero for each cell. At 4, 8, 12 and 24 h or 26 h, the complete receptor fluid was collected and replaced with fresh temperature equilibrated receptor medium. The determination of the diffused active ingredient concentration was determined from aliquots. At the end of the 24- or 26 h period, the skin surface was washed 5 times with 100 ml of distilled water or ethanol. The washing fluids were pooled and an aliquot part (1 ml) was assayed for the active ingredient concentration.

The cells were then dismantled and the dermis separated from the epidermis by means of elevated temperature as described herein. The active ingredient content was determined by means of HPLC or other validated analytical techniques. Furthermore, the presence of lactic or glycolic acid in the receptor medium was examined.

4. DiD Loaded PLGA NPs and NCs and/or Rhodamine PLGA Conjugated NPs or NCS Site Localization:

Excised human skin or porcine ear skin samples were placed on Franz diffusion cells (PermeGear, Inc., Hellertown, Pa.), with an orifice diameter of 5/11.28 mm, 5/8 mL receptor volume and an effective diffusion area of 0.2/1.0 cm². The receptor fluid was phosphate buffer, at pH 7.4. Throughout the experiment, the receptor chamber content was continuously agitated by a small magnetic stirrer. The temperature of the skin was maintained at 32° C. by water circulating system regulated at 37° C. The solutions and different NP and NCs formulations, either loaded with entrapped DiD fluorescent probe with free PLGA, or PLGA covalently bound to rhodamine B, were applied on the skin as detailed below. This protocol was adopted to follow the skin localization of both the entrapped DiD probe and of the conjugated rhodamine B polymer. The various formulations were prepared as described in the experimental section above. The dose applied for each formulation on the excised skin samples was 125 μl of a 30 mg/ml PLGA polymer concentration with an initial entrapped fluorescent content of DiD 30 μg/ml.

After single incubation period or at different time intervals, some of the skin samples were dissected to identify the localization site of the nanocarrier in the various skin layers by confocal microscope. The procedure was as follows using histological sectioning: the skin specimens were fixated using formaldehyde 4% for 30 minutes. The fixated tissues were placed in an adequate plastic cubic embedding in tissue freezing medium (OCT, Tissue-Tek). Skin samples were then deeply frozen at −80° C. and vertically cut into 10 μm thick sections, utilizing Cryostat at −20° C. Then, the treated specimens were stored in a refrigerator up to the confocal microscopic analysis.

In addition, some whole mount skin specimens were kept intact after Franz cells incubation at selected time interval of 2 h and immediately observed by confocal microscope, and further reconstructed using 3D imaging from z-stacks pictures. The fluorescence intensity versus skin depth for nanocarriers and respective controls using line profile was analyzed (calculated intensity for each section and whole specimen accumulative intensity). Samples data is given in Table 3.

TABLE 3
Description of the composition of each formulation
topically applied with specific equivalent dose

| | | PLGA- | | | DiD eq. |
|---|---|---|---|---|---|
| | PLGA, | rhodamine B | Oil core | | dose |
| Formulation | mg/cm² | conjugated % | type in NCs, | Volume | Applied |
| Composition | (MW, kDa) | w/w from NPs | (μl) | applied, μl | μg |
| DiD NPs | 4.5 (4) | — | — | 150 | 1.125 |
| DiD NPs | 4.5 (50) | — | — | 150 | 1.125 |
| DiD NCs | 4.5 (4) | — | Octanoic | 150 | 1.125 |
| | | | acid (75) | | |
| DiD micellar | — | — | — | 150 | 1.125 |
| solution | | | | | |
| DiD | 3.75 (4) | 5 | — | 125 | 3.75 |
| incorporated | | | | | |
| rhodamine B | | | | | |
| conjugated | | | | | |
| PLGA NPs | | | | | |
| DiD | 3.75 (4) | 5 | MCT (113) | 125 | 3.75 |
| incorporated | | | | | |
| rhodamine B | | | | | |
| conjugated | | | | | |
| PLGA NCs | | | | | |
| Rhodamine B | 3.75 (NA) | | — | 125 | — |
| incorporated | | | | | |
| Latex NPs | | | | | |
| DiD and | — | 5 | — | 125 | 3.75 |
| rhodamine B | | | | | |
| conjugated | | | | | |
| PLGA aqueous | | | | | |
| dispersion | | | | | |
| DiD and | — | 5 | MCT (113) | 125 | 3.75 |
| rhodamine B | | | | | |
| conjugated | | | | | |
| PLGA oil | | | | | |
| containing | | | | | |
| aqueous | | | | | |
| dispersion | | | | | |

### 5. [³H]DHEA NCs Site Localization and Deep Skin Layer Localization:

[³H]DHEA NCs formulations were applied on the skin using the Franz cell diffusion system. [³H]DHEA localization in the various skin layers was determined by skin compartment dissection technique. Dermatome pig skin (600-800 μm thick) was mounted on Franz diffusion cells (Crown Glass, Sommerville, N.J., USA) with an effective diffusion area of 1 cm² and an acceptor compartment of 8 ml (PBS, pH 7.4). At different time intervals, skin compartment dissection was carried out to identify the localization site of the nanocarriers in the skin surface, upper corneocytes layers, epidermis, dermis and receptor cell. First, the remaining of the formulation were collected following serial washings to allow adequate recovery. Then, the skin surface was removed by adequate sequential tape-stripping, contributing the first strip to the donor compartment. The rest of the viable epidermis was separated from the dermis by means of heat elevated temperature, and then chemically dissolved by solvable digestion liquid. Finally the receptor fluids was also collected and further analyzed.

In addition, in an attempt to reveal quantitatively the biofate of the NCs and NPs in the various layers of the skin, 80 and 127 μCi [³H]Cholesteryl oleyl ether ([³H]COE) were either dissolved in the oil core of the NCs or entrapped in the nanomatrices of the NPs respectively. The radioactive tracer, [³H]Cholesteryl oleyl ether ([³H]COE) is highly lipophilic with a log P above 15 (>15) and his localization within skin layers should reflect the localization of either the oil core of the NC and the nanomatrix of the NP since the probe cannot be released from the nanocarriers in view of this extremely high lipophilicity.

### 6. Oleylcysteineamide Synthesis and Characterization Oleylcysteineamide Synthesis

Under a flow of nitrogen the flask was charged via syringe with oleic acid (OA) (2.0 g, 7.1 mmol), 60 ml of dry tetrahydrofuran, and triethylamine (0.5 ml, 7.1 mmol). Stirring was commenced, and the solution was cooled to an internal temperature of −15° C. using a dry ice-isopropyl alcohol bath at −5° to −10° C. Ethyl chloroformate (0.87 ml, 6.1 mmol) was added and the solution was stirred for 5 min. The addition of ethyl chloroformate results in an internal temperature rised to +8 to +10° C. and the precipitation of a white solid. Following the precipitation the continuously stirred mixture, still in the dry-isopropyl alcohol bath, was allowed to reach an internal temperature of −14° C. Cysteine (1.0 g, 8.26 mmol) dissolved in 5% Na₂CO₃ solution (10 ml) introduced into the flask via a syringe needle, was vigorously bubbled through the solution for 10 min with manual stirring: the internal temperature rise abruptly to 25° C. With the flask still in the cooling bath, stirring was continued for an additional 30 min, and the reaction mixture was stored in the freezer at −15° C. overnight. The slurry was stirred with tetrahydrofuran (100 ml) at room temperature for 5 min and amine salts were removed by suction filtration through a Büchner funnel. After the solids were rinsed with tetrahydrofuran (20 ml), the filtrate was passed through a plug of silica gel (25 g Merck 60 230-400 mesh) in a coarse porosity sintered-glass filter funnel with aspirator suction. The funnel was further washed with acetonitrile (100 ml) and the combined filtrates were evaporated (rotary evaporator) to give a viscous liquid.

Formation of oleylcysteineamide was confirmed by H-NMR (Mercury VX 300, Varian, Inc., CA, USA) and LC-MS (Finnigan LCQDuo, ThermoQuest, NY, USA).

### Oleylcysteineamide Characterization

¹H-NMR (CDCl₃, δ): 0.818, 0.848, 0.868, 0.871, 0.889, 1.247, 1.255, 1.297, 1.391, 1.423, 1.452, 1.621, 1.642, 1.968, 1.989, 2.008, 2.174, 2.177, 2.268, 2.2932.320, 2.348, 3.005, 3.054, 4.881, 5.316, 5.325, 5.335, 5.343, 5.353, 5.369, 6.516, 6.540, 7.259 ppm.

LC-MS: Peak at 384.42.

The analysis of the NMR confirms the formation of the linker oleylcysteineamide, while the LC-MS spectrum clearly corroborates the molecular weight of the product which is 385.6 g/mol.

### 7. Preparation and Characterization of Surface Activated Nanoparticles and Macromolecules Conjugation:

Nanoparticles were prepared using the well established interfacial deposition method [19]. The oleylcysteineamide linker molecule was dissolved in the organic phase containing the polymer dissolved in water soluble organic solvent. The organic phase was then added drop wise to the aqueous phase which contained a surfactant. The suspension was evaporated at 37° C. under reduced pressure to a final nanoparticulate suspension volume of 10 ml. A maleimide bearing spacer molecule (LC-SMCC) was reacted with the desired macromolecule at pH 8 for subsequent conjugation to the thiol moiety. The thiol activated NPs and the relevant maleimide bearing molecule were then mixed and allowed to react overnight under a nitrogen atmosphere. The following day, free unbound molecules were separated from the conjugated NPs using a dia-filtration method.

TABLE 4
Formulation composition

| Organic phase | Aqueous phase |
|---|---|
| Polymer | Solutol HS 15 |
| 300 mg | 100 mg |
| Oleyl cysteine | Water |
| 20 mg | 100 ml |
| Tween 80 | |
| 100 mg | |
| Acetone | |
| 50 ml | |

### Size and Zeta Potential Characterization:

The size and zeta potential of the various NPs were measured in water using a DTS zetasizer (Malvern, UK).

### Determination of the Conjugation Efficiency of the Various Macromolecules to NPs:

The conjugation efficiency of the macromolecules such as MAbs was determined using the calorimetric Bicinchoninic acid assay (BCA) for protein quantification (Pierce, Ill., USA).

It should be noted, that the same procedure disclosed herein has been used to link hyaluronic acid to the nanoparticles.

8. Incorporation of Nanoparticles into a Novel Anhydrous Cream

The advantages of dispersing the final product in anhydrous cream are significant. Increasing amounts (0.1-10%) of freeze-dried powders of the NPs and the NPs prepared were incorporated into a dry formulation for topical application, namely a formulation essentially free of water. The relative amounts of the ingredients comprising this cream are detailed in Table 5.

TABLE 5
relative amounts of ingredients of dry formulation

| Ingredient | Relative amont/100 |
|---|---|
| Dow corning 9040- | 40.0-50.0 |
| Cyclopentasiloxane (and) | |
| Dimethicone crosspolymer | |
| Dimethicone | 5.0-7.0 |
| Cyclopentasiloxane | 10.0-15.0 |
| Shin etsu KSG-16 | 20.0-35.0 |
| Dimethicone (and) | |
| Dimethicone/Vinyl | |
| dimethicone crosspolymer | |
| Boron Nitride | 0.3-0.70 |
| lauroyl Lysine Ajinomoto | 0.2-0.70 |
| hyaluronic acid MP 50000 | 0.1-0.40 |
| Palmitoyloligopeptide - | 0.05-0.3 |
| Biopeptide CL Sederma | |
| Palmitoyl tetrapeptide - N- | 0.05-0.3 |
| Palmitoyl-Rigin | |

### IV. Results

### Nanoparticle Formulation and Characterization

Fluorescent nanoparticles were prepared to facilitate visual detection of the nanoparticles. PLA was conjugated to the fluorescent Rhodamine B probe. The nanoparticles were then prepared as described in the experimental section above.

The results demonstrate a homogenous nanoparticle formulation. It was possible to see the nanoparticles owing to the fluorescence labeling with Rhodamine fluorophore at excitation/emission 560/580 nm. The nanoparticles exhibited a mean diameter of 52 nm and a Zeta potential value of −37.3 mV.

This technique was used to detect and identify the localization of the nanoparticles with time in the various layers of the skin following topical application.

### Cryo-TEM Visualization of PLGA Biodegradable NPs One Month Following Preparation

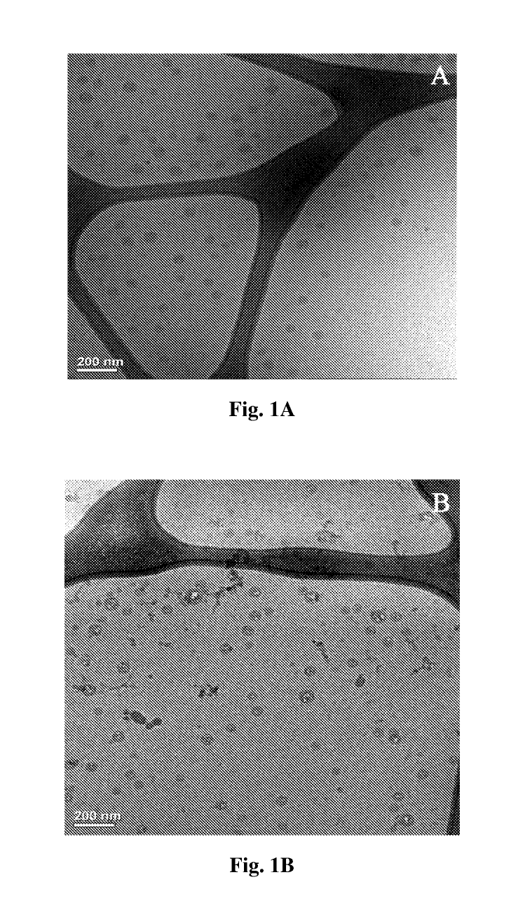

The Cryo-TEM images of blank PLGA₄₅₀₀ nanoparticles at various areas of the carbon grid are depicted in FIG. 1A. Nanoparticles appear quite homogenous in size and shape. Furthermore, cryo-TEM images of blank PLGA₄₅₀₀ nanoparticles at various areas of the carbon grid following one month storage at 4° C. are depicted in FIG. 1B. Nanoparticles were at different degradation stages. It can be noted that nanoparticles degraded with time in an aqueous environment.

### DHEA Loaded PLGA Nanoparticles

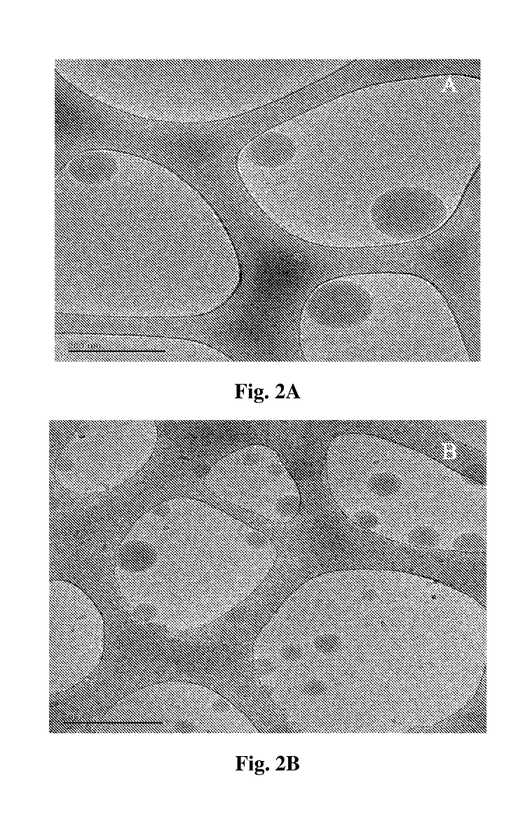

DHEA was encapsulated within the oil core of PLGA (4,500 or 50,000 Da) nanocapules. The Cryo-TEM images at various areas of the carbon grid are depicted in FIGS. 2A and B. The nanocapsules appear spherical and nanometric and no DHEA crystals were observed.

For encapsulation efficiency and active substance content determination, [³H] DHEA was incorporated within MCT NCs. The initial theoretical DHEA content for the cationic and anionic NCs, following diafiltration with PBS (pH 7.4), were 0.49 and 0.52%, while the observed contents were 0.18 and 0.15% respectively. The encapsulation efficiency was therefore 36.5 and 30.4% for the positively and negatively charged NCs, respectively (as shown in Table 6).

TABLE 6
DHEA content and loading efficiency within MCT NCs

| | Theoretical | Observed | |
|---|---|---|---|
| | conc. | conc. | Yield |
| Formulation | (%, w/v) | (%, w/v) | (%) |
| Positively charged [³H]DHEA loaded | 0.013 | 0.006 | 36.53 |
| MCT NCs | | | |
| Negatively charged [³H]DHEA | 0.013 | 0.005 | 30.40 |
| loaded MCT NCs | | | |

### Skin Penetration of Fluorescent Labeled Nanoparticles

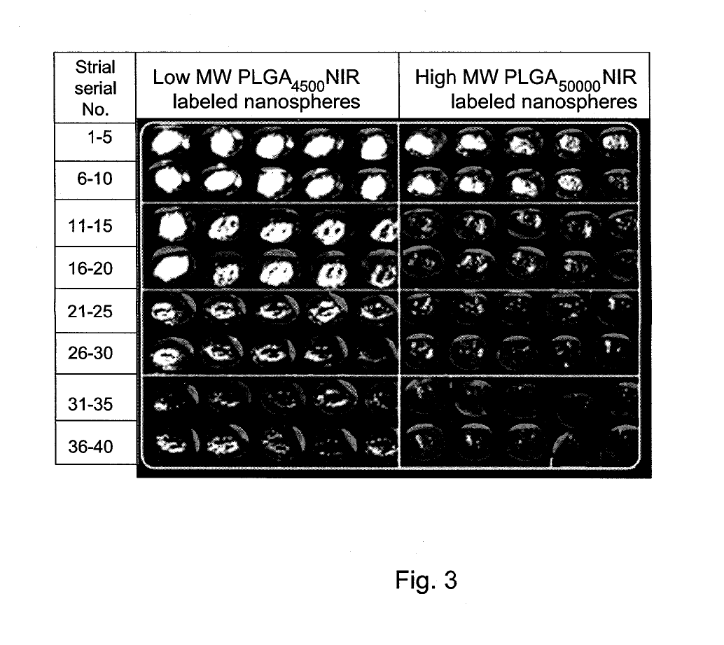

To evaluate skin penetration of NPs, nanospheres comprising of PLGA₄₅₀₀ or PLGA₅₀₀₀₀ were prepared, while a quantity of the polymer was covalently labeled with the infra-red dye NIR-783. Fluorescent formulations were topically administered on abdominal human skin of 60 years old male, using Franz cells (2.25 mg/cm²). Following 3 h, skin specimens were washed and scanned using ODYSSEY® Infra Red Imaging System (LI-COR Biosciences, NE, USA). Fluorescent images of various consecutive tape-stripping following topical administration are presented in FIG. 3. The results suggest that PLGA₄₅₀₀ penetrate deeper than PLGA₅₀₀₀₀ into the skin layers. Without being bound to theory, this may be attributed to the more rapid biodegradation of PLGA₄₅₀₀ compared to PLGA₅₀₀₀₀

### Skin Penetration of Fluorescent Labeled Nanocapsules

To evaluate skin penetration of nanocapsules (NCs), as compared to nanospheres (NSs), formulations were incorporated with the fluorescent probe DiD. In order to define the bio-fate of PLGA nanocarriers, DiD fluorescent-probe-loaded-MCT NCs coated with PLGA covalently bound to rhodamine B were prepared. In the absence of MCT, NPs were formed. Non-degradable commercially available rhodamine B loaded Latex nanospheres were also investigated.

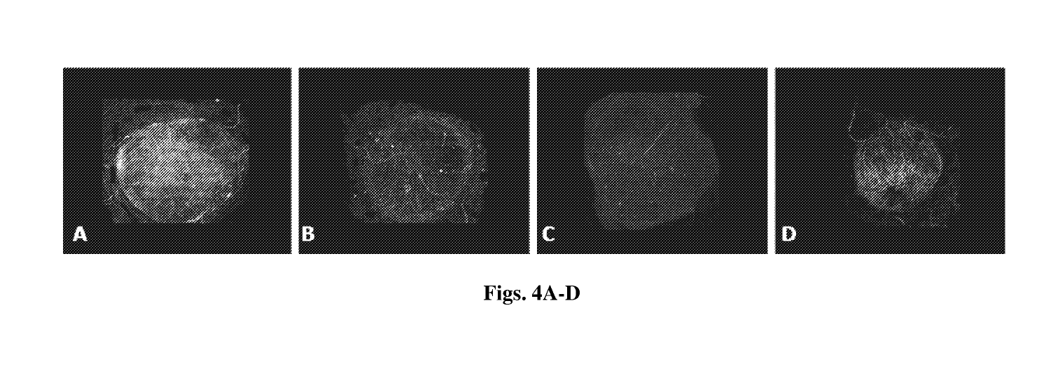

The fluorescent formulations were topically administered on abdominal human skin of 40 years old female, using Franz cells (4.5 mg/cm²). Following 2 h, skin specimens were washed and scanned using Zeiss LSM710 confocal laser scanning microscope. Reconstructed fluorescent images of whole skin specimens are depicted in FIGS. 4A-D. The results clearly indicate that all DiD loaded nanoparticles elicited larger fluorescent values as compared to DiD control solution. In addition, PLGA₄₅₀₀ nanocapsules exhibited superior skin penetration/retention as compared to other nanoparticulate delivery systems.

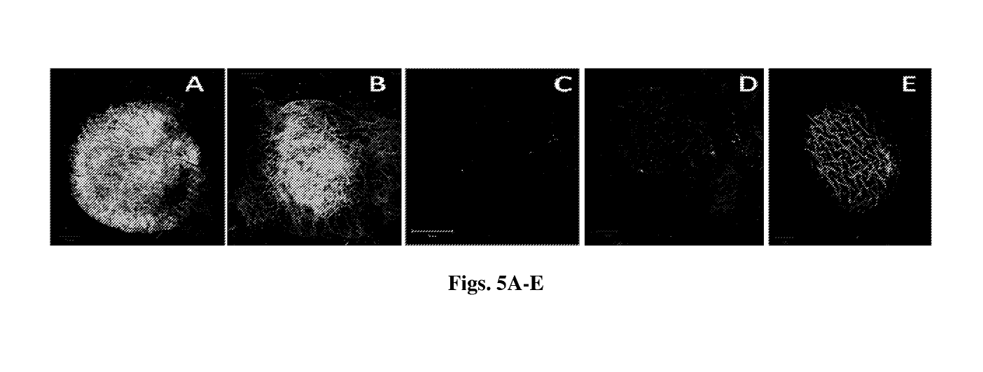

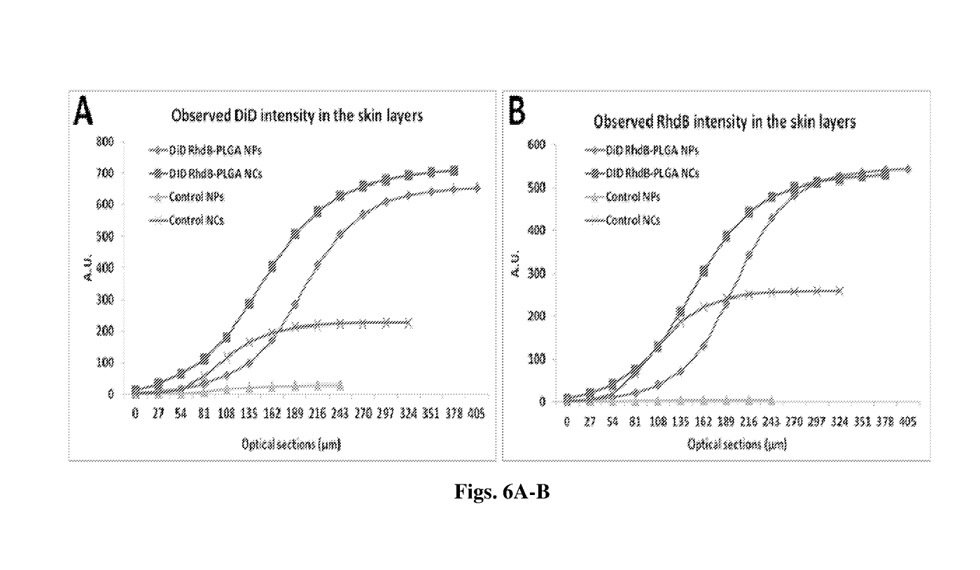

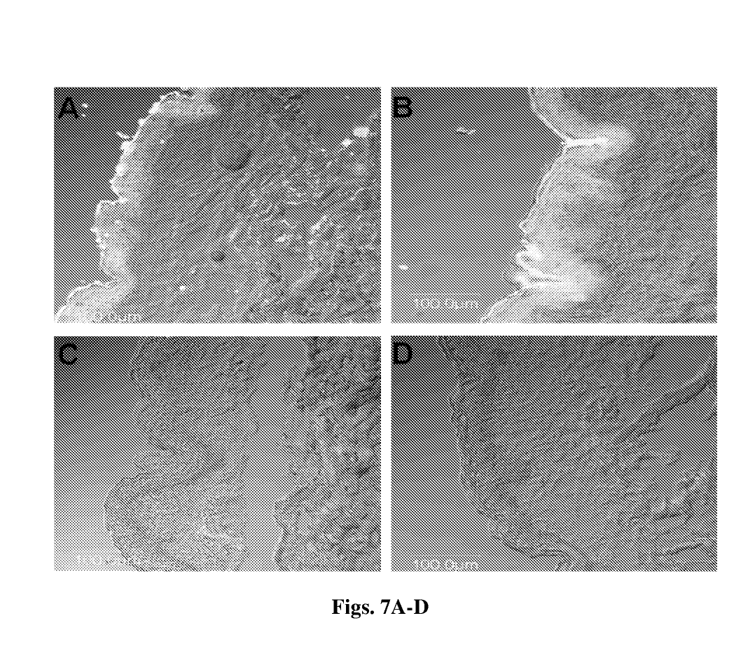

The dually labeled nanocarriers formulations and their respective controls were applied for 2 h on abdominal human skin of 50 years old female. Reconstructed fluorescent images of whole skin specimens are depicted in FIGS. 5A-E. The images of the NPs and NCs following a 2 h topical treatment show that more of the fluorescent cargo was released from NCs than NPs, although both reached the same depth in the skin (close to 200 μm), while the respective controls remained on the superficial skin layers. The results clearly indicate that DiD loaded nanoparticles penetrate at the same fashion as described above. Furthermore, rhodamine B fluorescence intensity, which is originally derived from the fluorescent probe conjugation to PLGA, was much higher when the PLGA based nanoparticulate carriers (i.e. nanocarriers) were topically administered (FIGS. 6A-B). This is also depicted in the cross section images (FIGS. 7A-D).

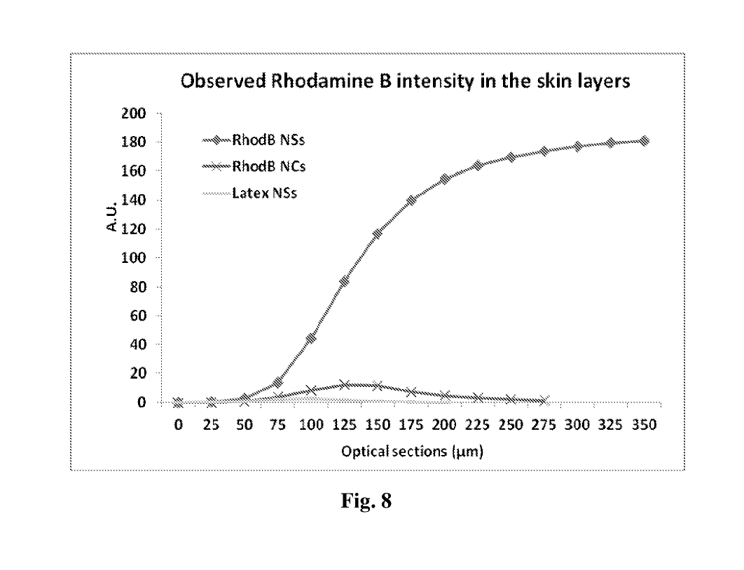

Finally, poor rhodamine B intensity was recorded following 2 h incubation of non-degradable rhodamine B latex NSs on abdominal human skin of 30 years female. This result suggests that non-degradable based carriers have an inferior ability to release their cargo when compared to degradable systems (FIG. 8).

### [³H]DHEA NCs Site Localization and Deep Skin Layer Localization

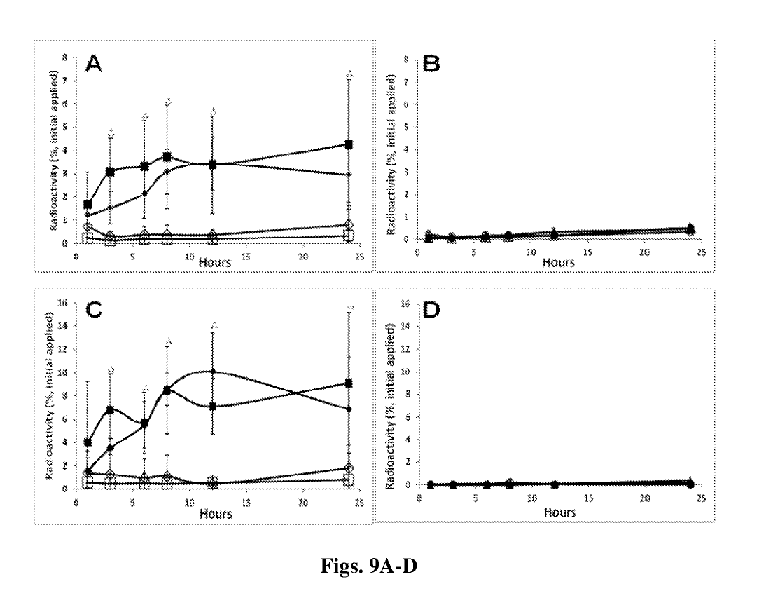

The results reported in FIGS. 9A-D show the ex-vivo dermato-biodistribution in the skin compartments of [³H]DHEA following topical application of negatively and positively charged [³H]DHEA loaded PLGA NCs and their respective controls at different incubation periods. Above 90% of the initial amount applied of the radiolabeled DHEA using different oil controls was recovered from the donor cell at each time interval up to 24 h. When DHEA loaded NCs were applied, again, most of the radioactive compound was collected at the donor compartment, with an average of over 90% up to 6 hours, with a notable decrease to approximately 80, 65 and 55% was recorded at 8, 12 and 24 hours, respectively. [³H]DHEA distribution in the upper skins layers as a function of SC (Stratum Corneum) depth following a sequential 10 tape-stripping tests (TS) is depicted in Table 7. Each pair of TS was extracted and analyzed by liquid scintillation, resulting in a sequence of five sub-layers description of the SC from each specimen. Regardless to the treatment applied, it can be noted that the highest levels of [³H]DHEA were detected in layers A and B, which represents the outermost layers of the SC, with a coordinate decrease recorded at the inner layers C, D and E. Time related accumulation of the radioactive compound in the different SC layers occurred when the negatively and positively charged [³H]DHEA loaded NCs were applied. It should be noted that irrespective of the formulation, the concentration of radioactivity within the SC was low (around 1-2%). It can clearly be observed that at 24 h post application, the concentration of radioactivity diminished progressively in the internal layers (Table 7) of the SC. However, marked differences between the DHEA loaded NCs and their respective controls were recorded in the viable skin compartments (epidermis and dermis). [³H]DHEA levels reached a plateau of ˜3% and 5.5% in the epidermis and dermis respectively, following 6 hours incubation of both positively and negatively charged DHEA NCs (FIG. 9), while [³H]DHEA levels obtained in the epidermis and dermis with the respective oil controls did not reach 1% over all the treatment periods up to 24 h (FIG. 9) (P<0.05).

TABLE 7
[³H]DHEA distribution over time in the different SC layers of porcine skin following
incubation with different nanocapsule formulations. Values are mean ± SD. N = 4

| | Incubation | | | | | |
|---|---|---|---|---|---|---|
| | periods | Stratum corneum layers (strips number) | | | | |
| Formulation | (hours) | A (1-2) | B (3-4) | C (5-6) | D (7-8) | E (9-10) |
| Positively | 1 | 0.2% ± 0.0 | 0.2% ± 0.1 | 0.1% ± 0.0 | 0.1% ± 0.1 | 0.1% ± 0.1 |
| charged | 3 | 0.3% ± 0.2 | 0.2% ± 0.1 | 0.1% ± 0.1 | 0.1% ± 0.1 | 0.1% ± 0.1 |
| [³H]DHEA | 6 | 0.3% ± 0.1 | 0.1% ± 0.1 | 0.1% ± 0.1 | 0.1% ± 0.1 | 0.1% ± 0.1 |
| loaded MCT NCs | 8 | 0.7% ± 0.6 | 0.3% ± 0.2 | 0.2% ± 0.1 | 0.1% ± 0.1 | 0.1% ± 0.1 |
| | 12 | 2.0% ± 1.8 | 0.8% ± 0.7 | 0.3% ± 0.2 | 0.3% ± 0.2 | 0.2% ± 0.1 |
| | 24 | 1.9% ± 0.9 | 0.8% ± 0.1 | 0.6% ± 0.2 | 0.4% ± 0.1 | 0.3% ± 0.1 |
| Negatively | 1 | 1.3% ± 0.1 | 0.4% ± 0.1 | 0.2% ± 0.1 | 0.1% ± 0.1 | 0.1% ± 0.0 |
| charged | 3 | 0.3% ± 0.0 | 0.2% ± 0.0 | 0.1% ± 0.0 | 0.1% ± 0.0 | 0.1% ± 0.0 |
| [³H]DHEA | 6 | 0.2% ± 0.1 | 0.2% ± 0.0 | 0.1% ± 0.0 | 0.1% ± 0.0 | 0.1% ± 0.0 |
| loaded MCT NCs | 8 | 0.8% ± 0.8 | 0.3% ± 0.3 | 0.3% ± 0.1 | 0.2% ± 0.1 | 0.2% ± 0.1 |
| | 12 | 1.9% ± 1.3 | 0.8% ± 0.3 | 0.4% ± 0.1 | 0.3% ± 0.2 | 0.3% ± 0.2 |
| | 24 | 2.9% ± 1.8 | 1.4% ± 0.5 | 0.7% ± 0.3 | 0.5% ± 0.3 | 0.4% ± 0.2 |
| Positively | 1 | 1.5% ± 0.9 | 1.1% ± 1.1 | 0.4% ± 0.5 | 0.2% ± 0.1 | 0.2% ± 0.1 |
| charged oil | 3 | 3.4% ± 1.4 | 1.4% ± 0.7 | 0.5% ± 0.3 | 0.2% ± 0.1 | 0.2% ± 0.1 |
| control | 6 | 2.4% ± 0.8 | 0.8% ± 0.3 | 0.3% ± 0.1 | 0.3% ± 0.1 | 0.2% ± 0.1 |
| | 8 | 1.5% ± 0.5 | 0.7% ± 0.3 | 0.3% ± 0.1 | 0.2% ± 0.1 | 0.1% ± 0.1 |
| | 12 | 4.6% ± 1.7 | 1.4% ± 0.6 | 0.5% ± 0.2 | 0.3% ± 0.1 | 0.2% ± 0.1 |
| | 24 | 2.7% ± 0.8 | 0.9% ± 0.3 | 0.5% ± 0.2 | 0.3% ± 0.2 | 0.2% ± 0.2 |
| Negatively | 1 | 2.2% ± 2.4 | 0.8% ± 0.7 | 0.2% ± 0.2 | 0.1% ± 0.0 | 0.1% ± 0.1 |
| charged oil | 3 | 1.7% ± 0.7 | 0.5% ± 0.2 | 0.2% ± 0.1 | 0.1% ± 0.1 | 0.1% ± 0.0 |
| control | 6 | 1.1% ± 0.3 | 0.3% ± 0.0 | 0.1% ± 0.1 | 0.1% ± 0.1 | 0.1% ± 0.0 |
| | 8 | 1.3% ± 0.1 | 0.4% ± 0.1 | 0.2% ± 0.1 | 0.1% ± 0.1 | 0.1% ± 0.0 |
| | 12 | 1.0% ± 0.5 | 0.2% ± 0.1 | 0.1% ± 0.0 | 0.1% ± 0.0 | 0.0% ± 0.0 |
| | 24 | 2.0% ± 0.6 | 0.7% ± 0.2 | 0.3% ± 0.1 | 0.2% ± 0.1 | 0.1% ± 0.1 |

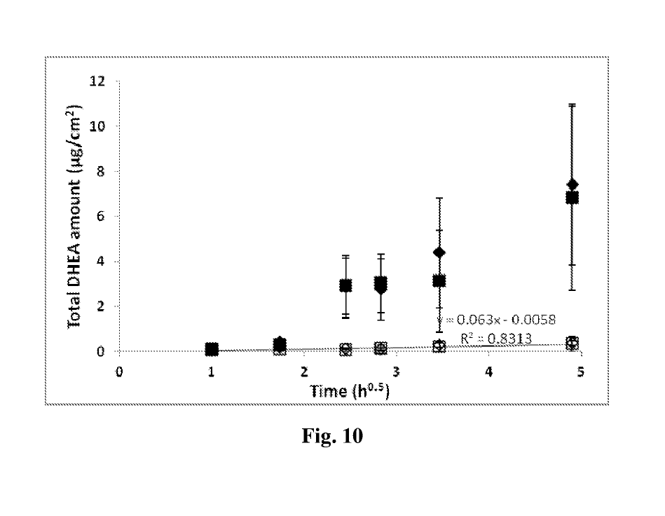

Increasing levels of the radioactive DHEA were found over time in the receptor compartment fluids when both positively and negatively DHEA loaded NCs were incubated, reaching 0.5%, 2.5% and 14% from the initial dose applied following 1 hour, 8 and 24 hours, respectively. On the other hand, the respective oil controls exhibited constant [³H]DHEA levels lower than 1% radioactivity at most time intervals. Although lag time of 3 hours was observed for the different formulations, [³H]DHEA appearance in the receptor fluids following positively and negatively NCs application was significantly higher than from the respective oil controls. The total amount of DHEA in the receptor fluids (μg/cm²), released from the different treatments, was plotted against the square root of time (FIG. 10). The low slow flux value 0.063 (μg/cm²/h^{0.5}), calculated from the slopes of the plotted graphs, for the oil controls correlates with their reported limited release profile. Then again, significant higher [³H]DHEA levels recorded in the receptor fluids when the negatively and positively DHEA NCs were topically applied, underlines a superior flux and superior percutaneous permeation of the DHEA when loaded into nanocarriers formulation. It should be emphasized that no significant difference between the two NCs formulation was observed at all time points, indicating that the nature of the charge did not contribute to the enhanced skin penetration but rather the type of nanostructure used, i.e. vesicular nanocapsules.

The highly lipophilic radioactive compound, [³H]COE, was incorporated into PLGA NSs and MCT containing NCs, in an attempt to identify the fate of the empty nanocarrier when topically applied. Following diafiltration with PBS (pH=7.4), the encapsulation efficiency was 45% and 70% for the NSs and the NCs, respectively. Aqueous and oil controls of [³H]COE, without polymer, were prepared for the ex-vivo experiments. Again, over 90% from the initial amount of the tritiated COE were collected from the donor compartment following each incubation period, irrespective of the formulation type (data not shown). Table 8 exhibits [³H]COE dermato-biodistribution as a function of the SC layers following the different treatments, as was previously described for [³H]DHEA. Up to 8 hours incubation of [³H]COE loaded NSs and NCs, less than 1% from the applied dose were extracted from the upper skin layers. Interestingly, a notable increase in layers A and B was observed following 12 hours incubation of the NSs and NCs. Although no notable differences in the levels of [³H]COE, associated to the incubation periods, were recorded when the different controls were topically applied, the constant distribution of the [³H]COE in MCT was higher in comparison to the [³H]COE surfactant solution (Table 8). Finally, less than 0.5% of radioactivity was counted in the viable compartments (epidermis, dermis and receptor fluids) during the incubation periods, when both nanocarriers formulations and their respective control were applied (FIG. 9). It appears that more incubation time is needed to differentiate between the various formulations of COE.

TABLE 8
[³H]COE distribution over time in the different SC layers of porcine skin following
incubation with different nanocapsules formulations. Values are mean ± SD. N = 3

| | Incubation | | | | | |
|---|---|---|---|---|---|---|
| | periods | Stratum corneum layers (strips number) | | | | |
| Formulation | (hours) | A (1-2) | B (3-4) | C (5-6) | D (7-8) | E (9-10) |
| [³H]Cholesteryl | 1 | 0.7% ± 0.8 | 0.2% ± 0.2 | 0.1% ± 0.1 | 0.1% ± 0.1 | 0.1% ± 0.1 |
| oleyl ether | 3 | 0.2% ± 0.1 | 0.2% ± 0.1 | 0.1% ± 0.1 | 0.1% ± 0.0 | 0.1% ± 0.0 |
| loaded PLGA NSs | 6 | 0.3% ± 0.2 | 0.2% ± 0.2 | 0.1% ± 0.1 | 0.1% ± 0.1 | 0.1% ± 0.1 |
| | 8 | 0.9% ± 1.0 | 0.3% ± 0.4 | 0.1% ± 0.1 | 0.1% ± 0.1 | 0.1% ± 0.1 |
| | 12 | 0.9% ± 1.3 | 0.4% ± 0.5 | 0.4% ± 0.5 | 0.2% ± 0.2 | 0.1% ± 0.2 |
| | 24 | 3.6% ± 0.7 | 1.5% ± 0.8 | 0.9% ± 0.5 | 0.7% ± 0.4 | 0.5% ± 0.4 |
| [³H]Cholesteryl | 1 | 0.2% ± 0.2 | 0.1% ± 0.0 | 0.1% ± 0.0 | 0.0% ± 0.0 | 0.0% ± 0.0 |
| oleyl ether | 3 | 0.4% ± 0.5 | 0.1% ± 0.1 | 0.1% ± 0.0 | 0.0% ± 0.0 | 0.0% ± 0.0 |
| loaded PLGA NCs | 6 | 0.4% ± 0.6 | 0.1% ± 0.1 | 0.2% ± 0.3 | 0.1% ± 0.1 | 0.0% ± 0.0 |
| | 8 | 0.6% ± 0.7 | 0.2% ± 0.2 | 0.1% ± 0.2 | 0.1% ± 0.1 | 0.1% ± 0.1 |
| | 12 | 1.2% ± 0.8 | 0.4% ± 0.4 | 0.2% ± 0.1 | 0.2% ± 0.2 | 0.1% ± 0.1 |
| | 24 | 2.4% ± 1.8 | 0.8% ± 0.6 | 0.4% ± 0.3 | 0.3% ± 0.2 | 0.2% ± 0.2 |
| [³H]Cholesteryl | 1 | 0.4% ± 0.8 | 0.2% ± 0.3 | 0.2% ± 0.3 | 0.1% ± 0.2 | 0.3% ± 0.6 |
| oleyl ether | 3 | 0.1% ± 0.1 | 0.1% ± 0.1 | 0.1% ± 0.0 | 0.0% ± 0.0 | 0.0% ± 0.0 |
| surfactant | 6 | 0.2% ± 0.1 | 0.1% ± 0.1 | 0.1% ± 0.0 | 0.1% ± 0.0 | 0.1% ± 0.0 |
| solution | 8 | 0.5% ± 0.5 | 0.3% ± 0.3 | 0.3% ± 0.3 | 0.1% ± 0.2 | 0.1% ± 0.1 |
| | 12 | 0.5% ± 0.4 | 0.3% ± 0.2 | 0.2% ± 0.2 | 0.1% ± 0.1 | 0.1% ± 0.1 |
| | 24 | 0.5% ± 0.1 | 0.3% ± 0.2 | 0.2% ± 0.1 | 0.2% ± 0.1 | 0.1% ± 0.1 |
| [³H]Cholesteryl | 1 | 1.8% ± 0.5 | 0.6% ± 0.4 | 0.3% ± 0.2 | 0.1% ± 0.1 | 0.1% ± 0.0 |
| oleyl ether oil | 3 | 1.0% ± 0.7 | 0.4% ± 0.3 | 0.1% ± 0.1 | 0.1% ± 0.0 | 0.0% ± 0.0 |
| control | 6 | 1.2% ± 0.3 | 0.4% ± 0.2 | 0.1% ± 0.0 | 0.1% ± 0.0 | 0.1% ± 0.0 |
| | 8 | 1.7% ± 0.5 | 0.8% ± 0.5 | 0.3% ± 0.1 | 0.1% ± 0.1 | 0.1% ± 0.0 |
| | 12 | 1.2% ± 0.5 | 0.7% ± 0.2 | 0.2% ± 0.1 | 0.1% ± 0.1 | 0.1% ± 0.1 |
| | 24 | 1.6% ± 0.3 | 0.5% ± 0.3 | 0.3% ± 0.1 | 0.1% ± 0.1 | 0.1% ± 0.0 |

### Hyaluronic Acid-Nanoparticles Conjugates

Finally, hyaluronic acid (HA) was conjugated, i.e. surface-associated, to the polymeric nanoparticles using the procedure for conjugation of the macromolecules. In brief, thiol activated NPs and a maleimide bearing HA (300000 Da MW) were prepared separately, then mixed and allowed to react overnight under a nitrogen atmosphere. Then, HA NPs were purified from the unconjugated HA using Vivaspin 300,000 MWCO diafiltration device (Vivascience, Stonehouse, UK).

The mean size of the resulting HA nanoparticles and the mean zeta potential were 184±30 nm (−73)±(−8) mV, respectively (n=9). It should be emphasized that the mean zeta potential of blank NPs is around −30 mV and the increase in the negative charge of the NPs when HA conjugated correlates to the polyanionic nature of the conjugated glycosaminoglycan.

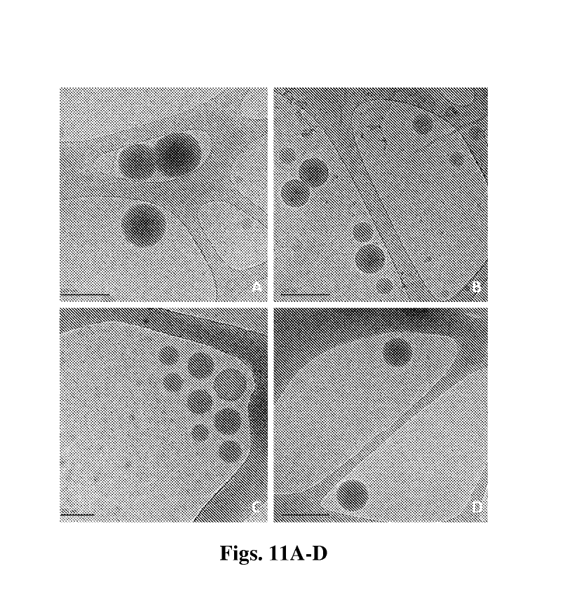

The CRYO-TEM images presented in FIG. 11 confirm the formation of spherical, nanometric and homogenous dispersion.

The HA amount conjugated to NPs was determined using modified DPA (diphenylamine) assay for the determination of sugars [20]. In brief, calibration curve and samples were incubated over 3 h at 110° C. with diphenylamine in the presence of trichloroacetic acid and sulfuric acid. The absorbance was determined at 530 nm using Synergy-HT Bio-Tek, Microplate Reader (BioTek Instruments). HA300 KDa was activated using two different cross-linkers: LC-SMCC and sulfo-SMCC, and then conjugated to SH bearing NPs. HA concentrations following activation via LC-SMCC(N-succinimidyl-6-[[4-(maleimidomethyl)cyclohexyl]carboxamido]caproate) and sulfo-SMCC were 298±43 and 258±16 μg/ml, respectively, and no significant difference was detected between the two cross-linkers. Therefore, the water soluble cross-linker sulfo-SMCC was further used.

An increase of the hyaluronic acid concentration conjugated to the surface of the nanoparticles by approximately 10 times fold was achieved and it may be as high as 15 mg/ml of hyaluronic acid surface decorated nanoparticles, resulting in an equivalent pure dose of 3.7±1.06 mg/ml hyaluronic acid.

### Nanoparticles and Nanocapsules Stability in Creams

The lack of stability of the particle of the invention in aqueous environments is one of their most important advantages, as the particles need to be biodegradable. For this purpose, a cosmetic silicone cream without the presence of water was prepared. As one of the major aspects is stability of the proposed nanoparticles in their cream formulations, stability studies of the nanoparticles in the cream have been performed.

Freeze-dried powders of nanoparticles and nanocapsules were dispersed in anhydrous cream (5% w/w) and were incubated under each of the following conditions:

Room temperature over 70 days

37° C. over 60 days, and then 10 days in RT

60° C. over 30 days, and then 10 days in RT

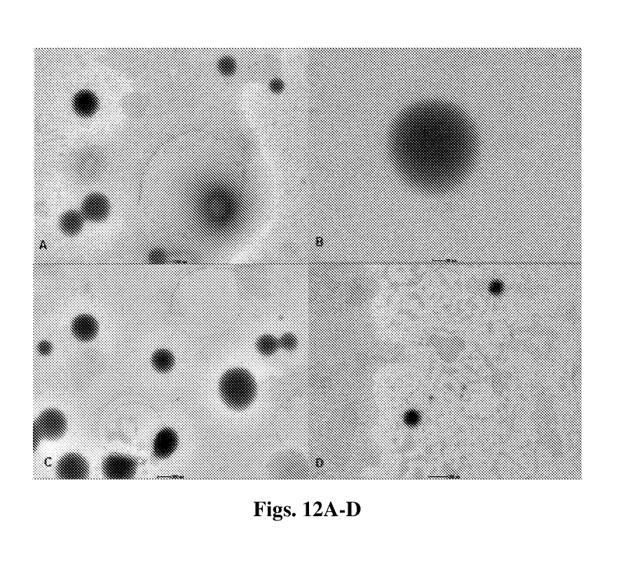

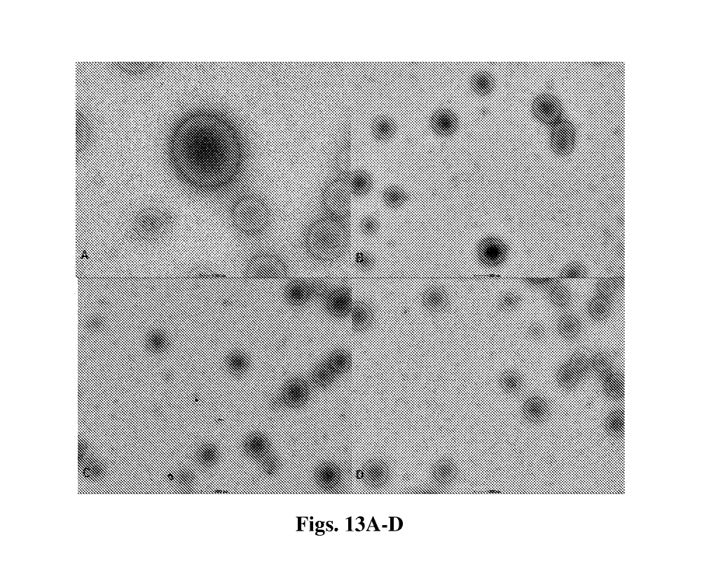

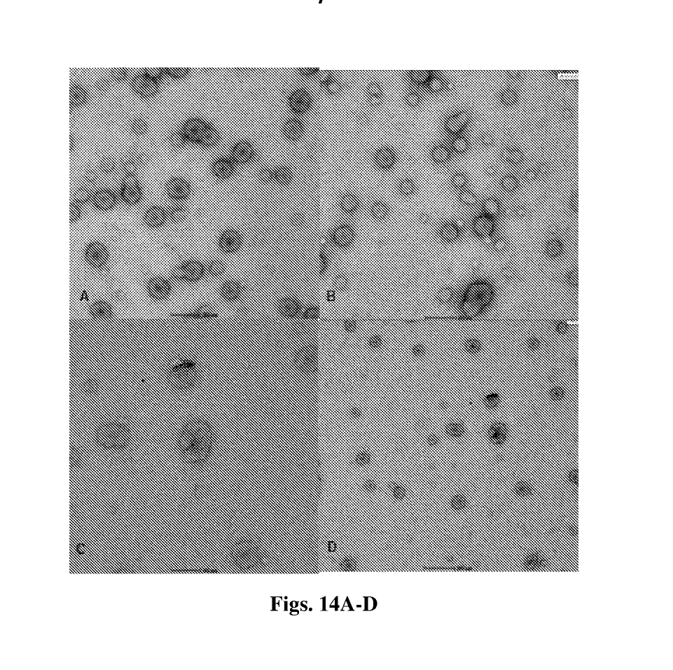

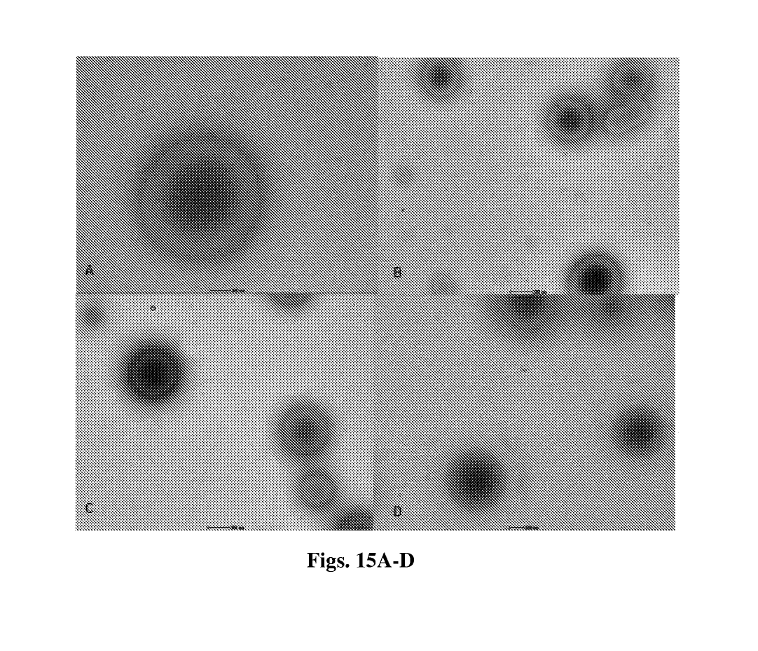

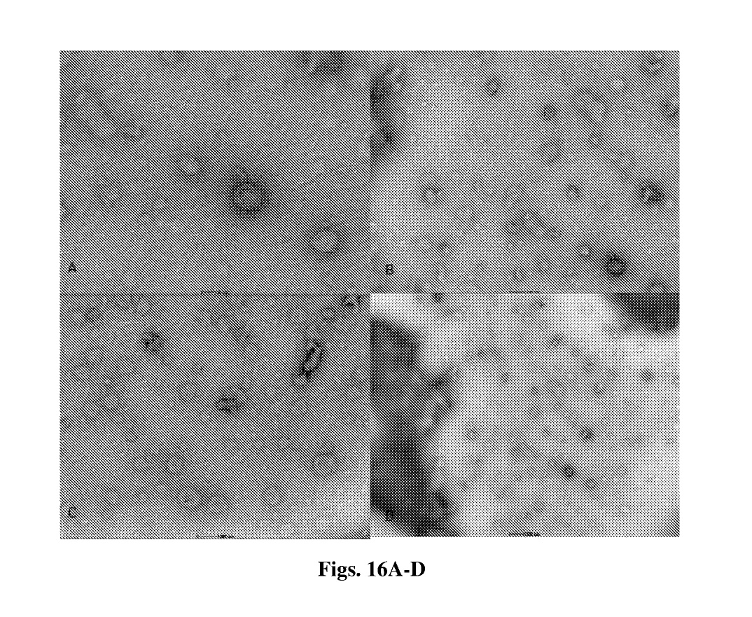

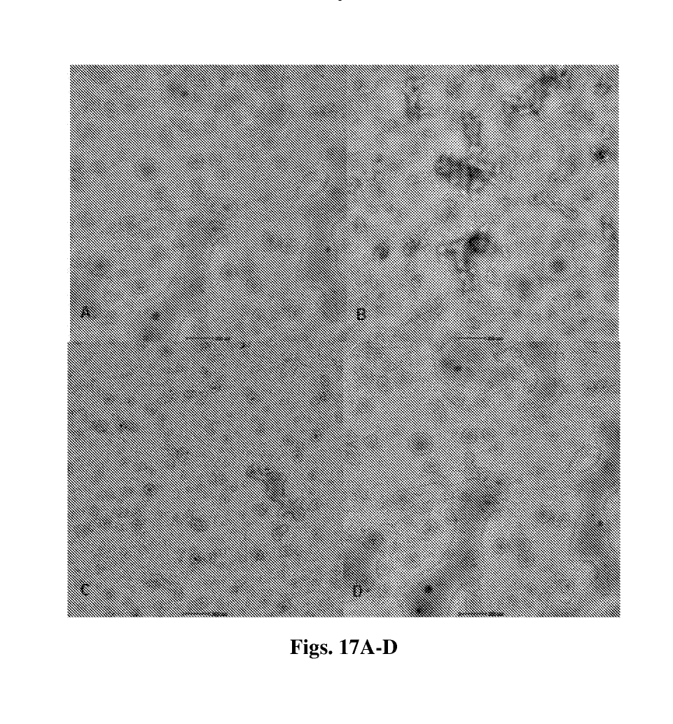

Nanoparticles were extracted from the creams and were depicted using transmission electron microscopy (TEM) with encouraging results. When the colloidal carriers dispersed in the cream were incubated at room temperature, both nanoparticles and nanocapsules (FIGS. 12 and 13, respectively) appeared complete, stable and homogenous. Incubation of nanocapsules at 37° C. showed similar results, while incubation of nanospheres at 37° C. exhibit minor degradation (FIGS. 14 and 15, respectively). Although both nanocarriers were present when incubated at 60° C., partial degradation was detected (FIGS. 16 and 17).

### NPs and NCs Scale-Up

Several steps in the fabrication of nanoparticles were adjusted to large scale manufacturing of various NCs, NPs and HA conjugated NPs.

The scaling-up development for manufacturing the nanoparticles and nanocapsules in large amounts resulted in elimination of several steps in the process:

1. Elimination of the acetone and/or water evaporation under reduced pressure using evaporator when nanoparticles were fabricated, did not change the mean size or the zeta potential of the NPs prior and following freeze-drying;
2. Two steps in the multi-step HA NPs conjugation processes were adjusted to large scale:
A. The cleaning procedure of the activated hyaluronic acid was eliminated. This procedure is time consuming and when eliminated the concentration of the hyaluronic acid conjugated to nanoparticles did not alter.
B. Freeze drying of activated hyaluronic acid prior to conjugation was efficient. This prevents the complex preparation of activated HA and activated NPs simultaneously.

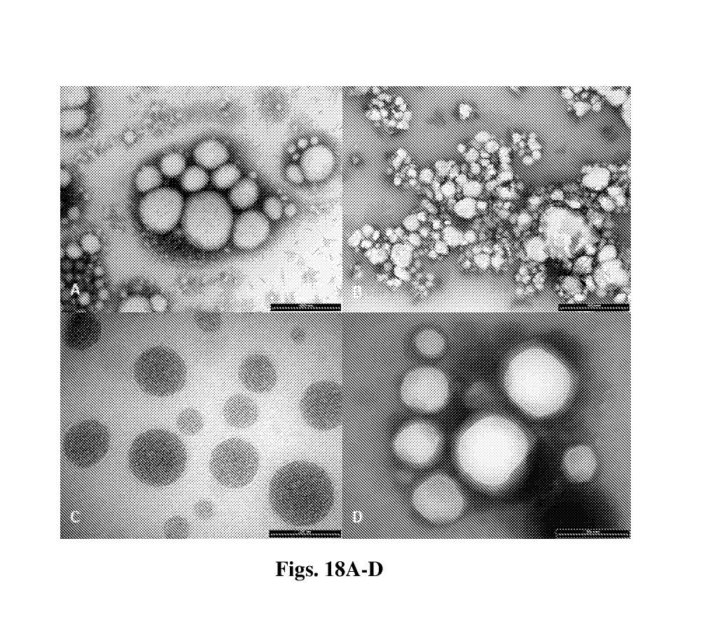

The feasibility of scaling of MCT NCs (FIG. 18A), oleic acid NCs (FIG. 18B), NPs (FIG. 18C) and HA NPs (FIG. 18D) prepared in large scale is demonstrated in the TEM micrograph.

### Large Scale Freeze Dried HA NPs, NPs and DHEA NCs Powders Dispersed in Creams

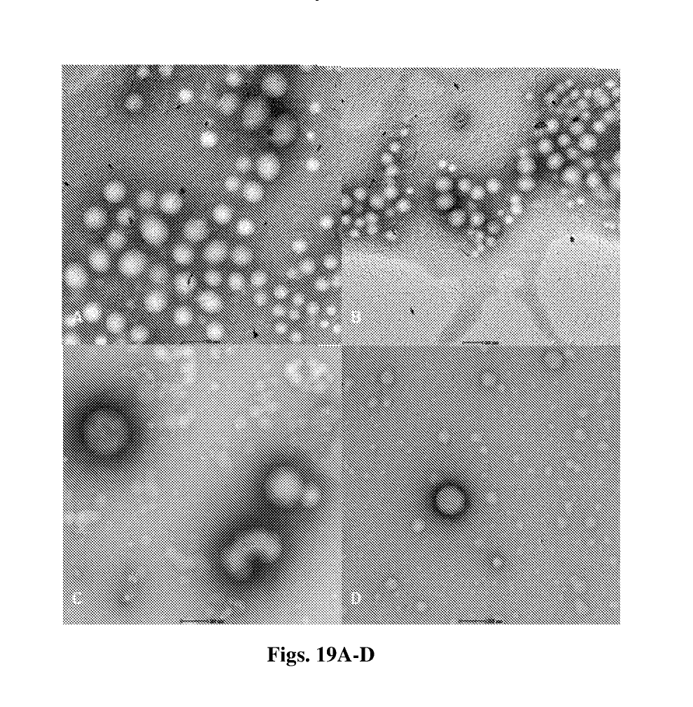

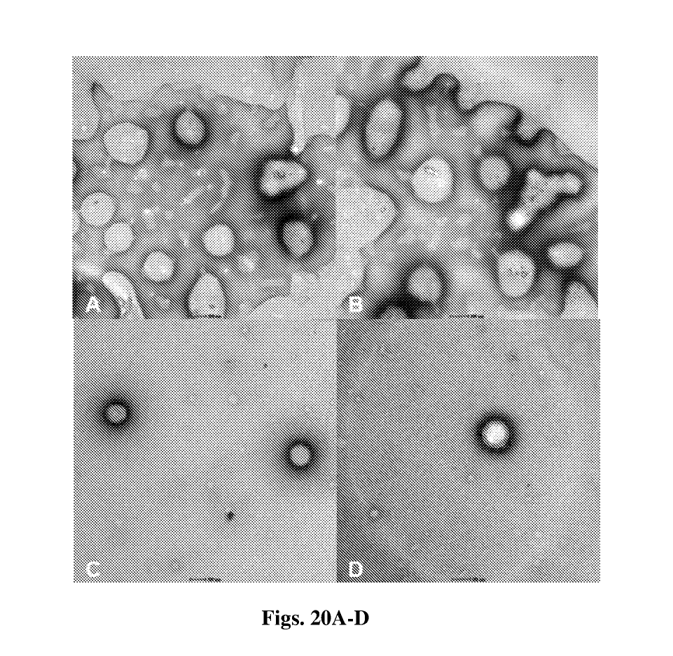

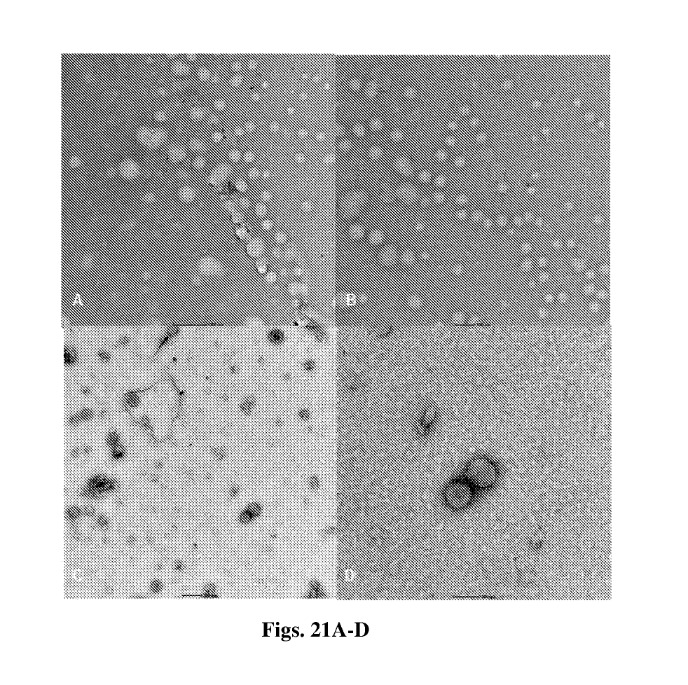

Nanoparticles prepared in large scale were freeze-dried and then, NPs powders were dispersed in anhydrous silicone cream as mentioned above. The TEM micrographs of the NPs and HA NPs powders following reconstitution with water are presented in FIGS. 19A-B and FIGS. 20A-B, respectively. The TEM micrographs of the extracted NPs and HA NPs powders following dispersion in anhydrous silicone cream are presented in FIGS. 19C-D and FIGS. 20C-D, respectively. Furthermore, DHEA NCs powders following reconstitution with water are presented in FIGS. 21A-B, while the TEM micrographs of the extracted DHEA NCs powders following dispersion in anhydrous silicone cream are presented in FIGS. 21C-D. The results clearly indicate that the large scale NPs powders are freely reinstituted upon dilution with water and are suitable for large manufacturing. Furthermore, these freeze-dried powders of colloidal carriers may be dispersed in anhydrous cream for various topical applications.

1. A cosmetic composition comprising poly(lactic glycolic)acid (PLGA) nanoparticles and a cosmetically acceptable carrier, the nanoparticles having an averaged diameter of at most 500 nm, the PLGA having an averaged molecular weight of between 2,000 and 20,000 Da.

2. The composition according to claim 1, wherein the nanoparticles are associated with at least one agent selected from the group consisting of a cosmetically-active agent and non-active agent.

3. (canceled)

4. The composition according to claim 2, wherein said at least one cosmetically-active agent is selected from the group consisting of a vitamin, a protein, an anti-oxidant, a peptide, a polypeptide, a lipid, a carbohydrate, a hormone, a prophylactic agent, a nutraceutical agent, a small molecule of a molecular weight of less than about 1,000 Da or less than about 500 Da, an electrolyte, a drug, a hydrophilic macromolecule, a lipophilic macromolecule, a macromolecule having molecular weight higher than 1.000 Da, and any combination of the aforementioned.

5-7. (canceled)

8. The composition according to claim 4, wherein the at least one cosmetically-active agent is a macromolecule selected from the group consisting of hyaluronic acid, collagen and DHEA.

9-11. (canceled)

12. The composition according to claim 2, wherein said non-active agent is selected to modulate one or more characteristic of the nanoparticle, said characteristic being selected from the group consisting of size, polarity, hydrophobicity/hydrophilicity, electrical charge, reactivity, chemical stability, clearance and targeting.

13. (canceled)

14. (canceled)

15. The composition according to claim 12, wherein the non-active agent is a fatty amino acid (alkyl amino acid).

16. (canceled)

17. (canceled)

18. The composition according to claim 2, wherein the at least one agent is associated with said nanoparticle via a chemical association selected from the group consisting of covalent bonding, electrostatic bonding, and hydrogen bonding, or via a physical association of at least a portion of the agent with the nanoparticle.

19. (canceled)

20. The composition according to claim 2, wherein the at least one agent is associated with the surface of said nanoparticle.

21. The composition of claim 20, wherein the at least one agent is at least one hydrophilic agent.

22. The composition according to claim 2, wherein the at least one cosmetically-active agent is associated with the surface of the nanoparticle via one or more linker moieties.

23. The composition according to claim 22, wherein said one or more linker moieties having a first portion capable of association with the nanoparticle and a second portion capable of association with the cosmetically-active agent.

24. (canceled)

25. (canceled)

26. The composition according to claim 23, wherein the linker is oleylcysteineamide.

27. (canceled)

28. (canceled)

29. The composition according to claim 2, wherein said at least one agent is associated to be contained within a core of said nanoparticle or within a matrix of said nanoparticle.

30. The composition according to claim 29, wherein said at least one agent is at least one lipophilic agent.

31. The composition according to claim 2, wherein at least one agent is associated with the surface of the nanoparticle and at least one different agent is associated to be contained within a core of said nanoparticle or within a matrix of said nanoparticle.

32-42. (canceled)

43. The composition according to claim 1, wherein the nanoparticles consist essentially of PLGA.

44. The composition according to claim 1, wherein the cosmetically acceptable carrier is a silicone-based carrier.

45-55. (canceled)

56. The composition according to claim 1, wherein the PLGA polymer is a copolymer of polylactic acid (PLA) and polyglycolic acid (PGA), the molar ratio of PLA to PGA being selected from the group consisting of 95:5, 90:10, 85:15, 80:20.75:25, 70:30, 65:35, 60:40, 55:45, and 50:50.

57-66. (canceled)

67. The composition of claim 1, being essentially free of water.

68. A polymeric nanoparticle having on its surface a plurality of cosmetically-active agents, each of said agents being associated to said nanoparticle via oleylcysteineamide.

69-71. (canceled)

72. A delivery system for topical application, the system comprising:
(i) a polymeric nanoparticle according to claim 68; and
(ii) at least one agent associated with said nanoparticle, said at least one agent being optionally associated with the nanoparticle surface via a linker moiety.

73-81. (canceled)
